(19) United States
(12) Patent Application Publication
(10) Pub. No.: US 2018/0110430 A1
(43) Pub. Date: Apr. 26, 2018

(54) Brain-Computer Interface

(71) Applicant: Clunbury Scientific LLC, Bloomfield Hills, MI (US)

(72) Inventor: Xueguo Zhang, Bloomfield Hills, MI (US)

(73) Assignee: Clunbury Scientific LLC, Bloomfield Hills, MI (US)

(21) Appl. No.: 15/334,977

(22) Filed: Oct. 26, 2016

(51) Int. Cl. A61B 5/04; G06N 3/06; A61B 5/00

(52) U.S. Cl. CPC A61B 5/04001; A61B 5/6868; G06N 3/061

(57) ABSTRACT

A brain-computer interface is disclosed. The brain-computer interface includes a spatially-adjustable animalia-engaging portion connected to a computing resource interface portion. The computing resource interface portion includes an actuator that is connected to at least one spatial-adjuster-component of the spatially-adjustable animalia-engaging portion. The actuator is configured to rotate the at least one spatial-adjuster-component for extending a distal portion of a length of a micro-electrode of at least one subassembly of the spatially-adjustable animalia-engaging portion beyond a distal end of a micro-electrode guide portion of the spatially-adjustable animalia-engaging portion.

### TECHNICAL FIELD

The disclosure relates to a brain-computer interface.

### BACKGROUND

A brain-computer interface (BCI), sometimes called a mind-machine interface (MMI), direct neural interface (DNI), or brain-machine interface (BMI), is generally a direct communication pathway between an enhanced or wired brain and an external device. BCIs are often directed at researching, mapping, assisting, augmenting, or repairing human cognitive or sensory-motor functions. While existing brain-computer interfaces perform adequately for their intended purpose, improvements to brain-computer interfaces are continuously sought in order to advance the arts.

### SUMMARY

One aspect of the disclosure provides a subassembly of a spatially-adjustable animalia-engaging portion of a brain-computer interface. The subassembly includes a micro-electrode-containing tube and a micro-electrode. The micro-electrode-containing tube is defined by a distal surface, a proximal surface, an outer surface and an inner surface. The micro-electrode-containing tube is defined by a length extending between the distal surface and the proximal surface. The inner surface of the micro-electrode-containing tube defines a micro-electrode-receiving passage having a passage diameter extending through the length of the micro-electrode-containing tube from the distal surface to the proximal surface. Access to the micro-electrode-receiving passage is provided by a distal opening formed by the distal surface of the micro-electrode-containing tube and a proximal opening formed by the proximal surface of the micro-electrode-containing tube. The outer surface of the micro-electrode-containing tube defines an outer diameter of the micro-electrode-containing tube.

The micro-electrode is disposed within the micro-electrode-receiving passage of the micro-electrode-containing tube. The at least one micro-electrode is defined by a distal tip, a proximal surface and an outer surface. The at least one micro-electrode is further defined by a length extending between the distal tip and the proximal surface of the micro-electrode.

The outer surface of the micro-electrode defines an outer diameter of the micro-electrode that is less than the passage diameter of the micro-electrode-containing tube. The proximal surface of the micro-electrode is substantially aligned with the proximal surface of the micro-electrode-containing tube. The distal tip of the micro-electrode is arranged beyond the distal surface of the micro-electrode-containing tube.

A portion of the body of the micro-electrode defined by the outer surface of the micro-electrode deviates from an axial extending through an axial center of both of the distal tip and the proximal surface of the micro-electrode whereby one or more portions of the portion of the body of the micro-electrode defined by the outer surface of the micro-electrode is disposed adjacent one or more portions of the inner surface defining the micro-electrode-receiving passage of the micro-electrode-containing tube for frictionally-fitting the micro-electrode within the micro-electrode-containing tube in an axially-adjustable orientation.

Implementations of the disclosure may include one or more of the following optional features. For example, the portion of the body of the micro-electrode extends along a proximal portion of the length of the micro-electrode from the proximal surface of the micro-electrode.

In some implementations, the portion of the body of the micro-electrode includes a sinusoidal shape that deviates from the axis. The one or more portions of the portion of the body of the micro-electrode may be defined by peaks of the sinusoidal shape.

In some examples, the micro-electrode is formed from one or more conductive filaments including one or more of a combination of a metal material and a non-metal material. The metal material may include one or more of stainless steel, carbon, tungsten, platinum and iridium. The non-metal material may include a conductive polymer.

In some implementations, the outer diameter of the micro-electrode ranges between approximately 12.5 μm-50 μm. The length of the micro-electrode may range between approximately 10 mm-40 mm.

Another aspect of the disclosure provides a spatially-adjustable animalia-engaging portion of a brain-computer interface. The spatially-adjustable animalia-engaging portion includes a micro-electrode retainer portion, a micro-electrode guide portion, at least one spatial-adjuster-component, at least one spatial-adjuster-guide-post, and at least one subassembly including a micro-electrode disposed within a micro-electrode-containing tube.

The micro-electrode retainer portion includes a distal end, a proximal end and a length extending between the distal end of the micro-electrode retainer portion and the proximal end of the micro-electrode retainer portion. The micro-electrode retainer portion further defines at least one spatial-adjuster-component-containing passage extending through the length of the micro-electrode retainer portion, at least one spatial-adjuster-guide-post passage extending through the length of the micro-electrode retainer portion, and at least one tube-and-micro-electrode-containing passage extending through the length of the micro-electrode retainer portion.

The micro-electrode guide portion includes a distal end, a proximal end and a length extending between the distal end of the micro-electrode guide portion and the proximal end of the micro-electrode guide portion. The micro-electrode guide portion further defines at least one spatial-adjuster-component-containing passage extending through the length of the micro-electrode guide portion, at least one spatial-adjuster-guide-post passage extending through the length of the micro-electrode guide portion, and at least one micro-electrode-containing passage extending through the length of the micro-electrode guide portion.

The at least one spatial-adjuster-component is disposed within the at least one spatial-adjuster-component-containing passage of each of the micro-electrode retainer portion and the micro-electrode guide portion. The at least one spatial-adjuster-guide-post is disposed within the at least one spatial-adjuster-guide-post passage of each of the micro-electrode retainer portion and the micro-electrode guide portion.

The at least one subassembly is disposed within the at least one tube-and-micro-electrode-containing passage of the micro-electrode retainer portion. An intermediate portion of the length of the micro-electrode of the at least one subassembly extends beyond the distal end of the micro-electrode retainer portion and is arranged within the at least one micro-electrode-containing passage of the micro-electrode guide portion. A distal portion of the length of the micro-electrode of the at least one subassembly extends beyond the distal end of the micro-electrode guide portion.

Implementations of the disclosure may include one or more of the following optional features. For example, a proximal portion of the length of the micro-electrode of the at least one subassembly extends between the distal end of the micro-electrode retainer portion and the proximal end of the micro-electrode guide portion.

In some implementations, the at least one spatial-adjuster-component-containing passage of the micro-electrode guide portion is defined by a threaded surface that is interfaced with an outer threaded surface of the at least one spatial-adjuster-component. The distal end of the micro-electrode retainer portion may be arranged in a spaced-apart opposing relationship with respect to the proximal end of the micro-electrode guide portion.

In some examples, the at least one spatial-adjuster-component-containing passage of each of the micro-electrode retainer portion and the micro-electrode guide portion are axially-aligned, wherein the at least one spatial-adjuster-guide-post passage of each of the micro-electrode retainer portion and the micro-electrode guide portion are axially-aligned. The at least one tube-and-micro-electrode-containing passage of the micro-electrode retainer portion may be axially-aligned with the at least one micro-electrode-containing passage of the micro-electrode guide portion.

In yet another aspect of the disclosure provides a computing resource interface portion of a brain-computer interface. The computing resource interface portion includes at least one interface subassembly and a micro-electrode retainer interface body portion. The at least one interface subassembly includes a distal biased pin, an intermediate biasing member and a proximal electrical contact. The micro-electrode retainer interface body portion is defined by a length extending between a distal end of the micro-electrode retainer interface body portion and a proximal end of the micro-electrode retainer interface body portion.

The micro-electrode retainer interface body portion includes an inner surface that defines at least one biased-pin-containing passage extending through the length of the micro-electrode retainer interface body portion. Access to the at least one biased-pin-containing passage is provided by a distal opening formed by the distal end of the micro-electrode retainer interface body portion and a proximal opening formed by the proximal end of the micro-electrode retainer interface body portion. The at least one interface subassembly is disposed within the at least one biased-pin-containing passage and arranged adjacent one or more portions of the inner surface of the micro-electrode retainer interface body portion.

Implementations of the disclosure may include one or more of the following optional features. For example, the distal biased pin includes a body extending between a distal end of the body of the distal biased pin and a proximal end of the body of the distal biased pin. The intermediate biasing member may include a body extending between a distal end of the body of the intermediate biasing member and a proximal end of the body of the intermediate biasing member. The distal end of the body of the intermediate biasing member may be disposed adjacent the proximal end of the body of the distal biased pin.

In some implementations, the proximal electrical contact includes a body extending between a distal end of the body of the proximal electrical contact and a proximal end of the body of the proximal electrical contact. The distal end of the body of the proximal electrical contact may be disposed adjacent the proximal end of the body of the distal biased pin.

In some examples, the proximal electrical contact is fixed adjacent the inner surface of the micro-electrode retainer interface body portion. A portion of a length of the proximal electrical contact may extend through the proximal opening and beyond the proximal end of the micro-electrode retainer interface body portion. The distal biased pin may be movably-disposed within the at least one biased-pin-containing passage. The intermediate biasing member may bias a shoulder surface of the distal biased pin adjacent a portion of the one or more portions of the inner surface of the micro-electrode retainer interface body portion defining a ledge surface such that a portion of a length of the distal biased pin extends through the distal opening and beyond the distal end of the micro-electrode retainer interface body portion.

In some implementations, the body of the distal biased pin defines an axial passage extending between the distal end of the body of the distal biased pin and the proximal end of the body of the distal biased pin. The body of the proximal electrical contact may define an axial passage extending between the distal end of the body of the proximal electrical contact and the proximal end of the body of the proximal electrical contact. The body of the intermediate biasing member may define an axial passage extending between the distal end of the body of the intermediate biasing member and the proximal end of the body of the intermediate biasing member. The axial passages may collectively define at least one micro-electrode access passage.

Another aspect of the disclosure provides a brain-computer interface. The brain-computer interface includes a spatially-adjustable animalia-engaging portion connected to a computing resource interface portion. The computing resource interface portion further includes an actuator that is connected to at least one spatial-adjuster-component of the spatially-adjustable animalia-engaging portion. The actuator is configured to rotate the at least one spatial-adjuster-component for further extending a distal portion of a length of the micro-electrode of at least one subassembly of the spatially-adjustable animalia-engaging portion beyond a distal end of a micro-electrode guide portion of the spatially-adjustable animalia-engaging portion.

Implementations of the disclosure may include one or more of the following optional features. For example, upon connecting the computing resource interface portion to the spatially-adjustable animalia-engaging portion, the distal end of the body of the distal biased pin of the interface subassembly of the computing resource interface portion is disposed adjacent at least one of a proximal surface of a micro-electrode-containing tube and a proximal surface of at least one micro-electrode of the at least one subassembly. As a result of the above-described arrangement, a distal end of the computing resource interface portion may be electrically connected to a proximal end of the spatially-adjustable animalia-engaging portion.

In some implementations, the proximal end of the body of the proximal electrical contact of the interface subassembly of the computing resource interface portion is connected to a conduit for connecting the computing resource interface portion to a computing resource. The conduit may be a wired conduit that hard-wire connects the computing resource interface portion to a computing resource. The conduit may be a wireless conduit that wirelessly connects the computing resource interface portion to a computing resource.

In some examples, the computing resource interface portion further includes at least one male connector portion extending away from the distal end of the body portion of the computing resource interface portion. The spatially-adjustable animalia-engaging portion may define at least one female connector portion extending into the proximal end of the micro-electrode retainer portion that is sized for receiving the at least one male connector portion for connecting the computing resource interface portion to the spatially-adjustable animalia-engaging portion.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a view of an exemplary BCI connecting an animal's brain to a computing resource.

FIG. 2 is a view of an exemplary BCI and a portion of a computing resource.

FIG. 3 is an enlarged portion of the BCI according to line 3 of FIG. 2.

FIG. 4A is a cross-sectional view of a micro-electrode guide portion of the BCI according to line 4A-4A of FIG. 3.

FIG. 4B is another cross-sectional view of the micro-electrode guide portion of the BCI according to line 4B-4B of FIG. 3.

FIG. 5A is a cross-sectional view of a micro-electrode retainer portion of the BCI according to line 5A-5A of FIG. 3.

FIG. 5B is another cross-sectional view of the micro-electrode retainer portion of the BCI according to line 5B-5B of FIG. 3.

FIG. 6 is a perspective view of a micro-electrode-containing tube of the BCI of FIG. 2.

FIG. 7 is a cross-sectional view of the micro-electrode-containing tube according to line 7-7 of FIG. 6.

FIG. 8 is a perspective view of a micro-electrode of the BCI of FIG. 2.

FIG. 9 is a cross-sectional view of the micro-electrode according to line 9-9 of FIG. 8.

FIG. 10 is a side view of a spatial-adjuster-component of the BCI of FIG. 2.

FIG. 11 is a side view of a spatial-adjuster-guide-post of the BCI of FIG. 2.

FIG. 12A is an exploded partial cross-sectional view of a first subassembly of the BCI of FIG. 2 including the micro-electrode guide portion of FIG. 4B, the micro-electrode retainer portion of FIG. 5B, the spatial-adjuster-component of FIG. 10 and the spatial-adjuster-guide-post of FIG. 11.

FIG. 12B is an assembled partial cross-sectional view according to FIG. 12A.

FIG. 13A is an exploded cross-sectional view of a second subassembly of the BCI of FIG. 2 including the micro-electrode-containing tube of FIG. 7 and the micro-electrode of FIG. 9.

FIG. 13B is an assembled cross-sectional view according to FIG. 13A.

FIG. 13C is an enlarged view according to line 13C of FIG. 13A.

FIG. 13D is an enlarged view according to line 13D of FIG. 13A.

FIG. 14A is an exploded partial cross-sectional view of a third subassembly of the BCI of FIG. 2 including the micro-electrode guide portion of FIG. 4A, the micro-electrode retainer portion of FIG. 5A and the second subassembly of FIG. 13B.

FIG. 14B is an assembled cross-sectional view according to FIG. 14A and referenced from line 14B-14B of FIG. 15A and line 14B-14B of FIG. 15B.

FIG. 15A is a top view of the third subassembly according to line 15A of FIG. 14B.

FIG. 15B is another top view of the third subassembly according to line 15B of FIG. 14B.

FIG. 16A is a perspective view of a distal biased pin of the BCI of FIG. 2.

FIG. 16B is a cross-sectional view of the distal biased pin according to line 16B-16B of FIG. 16A.

FIG. 16C is a perspective view of an intermediate biasing member of the BCI of FIG. 2.

FIG. 16D is a cross-sectional view of the intermediate biasing member according to line 16D-16D of FIG. 16C.

FIG. 16E is a perspective view of a proximal electrical contact of the BCI of FIG. 2.

FIG. 16F is a cross-sectional view of the proximal electrical contact according to line 16F-16F of FIG. 16E.

FIG. 16G is an exploded cross-sectional view of a subassembly of the BCI of FIG. 2 including the distal biased pin of FIG. 16B, the intermediate biasing member of FIG. 16D and the proximal electrical contact of FIG. 16E that is disposed within a micro-electrode retainer interface body portion referenced from line 16H-16H of FIG. 3.

FIG. 16H is an assembled cross-sectional view of the subassembly of FIG. 16G disposed within the micro-electrode retainer interface body portion according to line 16H-16H of FIG. 3 and a portion of third subassembly of FIG. 14B arranged in a spaced-apart relationship with respect to the micro-electrode retainer interface body portion.

FIG. 16I is cross-sectional view showing the portion of third subassembly of FIG. 14B arranged in an adjacent, connected relationship with respect to the micro-electrode retainer interface body portion according to FIG. 16H.

FIG. 16J is an assembled cross-sectional view according to FIG. 16I showing an electrical conduit connected to the proximal electrical contact of the subassembly that is disposed within the micro-electrode retainer interface body portion.

FIG. 16A′ is a perspective view of a distal biased pin of the BCI of FIG. 2.

FIG. 16B′ is a cross-sectional view of the distal biased pin according to line 16B′-16B′ of FIG. 16A′.

FIG. 16C′ is a perspective view of an intermediate biasing member of the BCI of FIG. 2.

FIG. 16D′ is a cross-sectional view of the intermediate biasing member according to line 16D′-16D′ of FIG. 16C′.

FIG. 16E′ is a perspective view of a proximal electrical contact of the BCI of FIG. 2.

FIG. 16F′ is a cross-sectional view of the proximal electrical contact according to line 16F′-16F′ of FIG. 16E′.

FIG. 16G′ is an exploded cross-sectional view of a subassembly of the BCI of FIG. 2 including the distal biased pin of FIG. 16B′, the intermediate biasing member of FIG. 16D′ and the proximal electrical contact of FIG. 16E′ that is disposed within a micro-electrode retainer interface body portion.

FIG. 16H′ is an assembled cross-sectional view of the subassembly of FIG. 16G′ disposed within the micro-electrode retainer interface body portion and a portion of third subassembly of FIG. 14B arranged in a spaced-apart relationship with respect to the micro-electrode retainer interface body portion.

FIG. 16I′ is cross-sectional view showing the portion of third subassembly of FIG. 14B arranged in an adjacent, connected relationship with respect to the micro-electrode retainer interface body portion according to FIG. 16H′.

FIG. 16J′ is an assembled cross-sectional view according to FIG. 16I′ showing an electrical conduit connected to the proximal electrical contact of the subassembly that is disposed within the micro-electrode retainer interface body portion.

FIG. 17 is a bottom view of the micro-electrode retainer interface body portion including the subassembly of FIG. 16G according to line 17 of FIG. 16H.

FIG. 18A is a partial view of the BCI of FIG. 2 having a plurality of micro-electrodes spatially arranged in a first orientation.

FIG. 18B is a partial view of the BCI of FIG. 2 having the plurality of micro-electrodes spatially adjusted to a second orientation from the first orientation of FIG. 18A.

FIG. 18C is a partial view of the BCI of FIG. 2 having most of the plurality of micro-electrodes spatially arranged in the second orientation of FIG. 18B and at least one micro-electrode of the plurality of micro-electrodes spatially adjusted to a third orientation.

FIG. 19A is an enlarged view of the BCI according to line 19 of FIG. 18C.

FIG. 19B is a further enlarged view according to FIG. 19A showing a push-pin being utilized for spatially adjusting the at least one micro-electrode of the plurality of micro-electrodes to the third orientation.

FIG. 19C is another view of the BCI according to FIG. 19B after the at least one micro-electrode of the plurality of micro-electrodes is spatially adjusted to the third orientation.

FIG. 20A is an enlarged view of the BCI referenced from line 19 of FIG. 18C but including the subassembly of FIG. 16G′.

FIG. 20B is a further enlarged view according to FIG. 20A showing a push-pin being utilized for spatially adjusting the at least one micro-electrode of the plurality of micro-electrodes to the third orientation.

FIG. 20C is another view of the BCI according to FIG. 20B after the at least one micro-electrode of the plurality of micro-electrodes is spatially adjusted to the third orientation.

Like reference symbols in the various drawings indicate like elements.

### DETAILED DESCRIPTION

The figures illustrate an exemplary implementation of a brain-computer interface (BCI). When interfaced with an animal, the BCI includes structure that permits selective spatial adjustment of one or more micro-electrodes into the animalia's brain at a desired depth. After the BCI is interfaced with the animalia's brain, the BCI may be utilized for measuring brain signals of brain neurons of the animalia's brain for the purpose of, for example, conducting research on the brain function of the animalia or assisting and/or repairing cognitive or sensory-motor functions of the animalia.

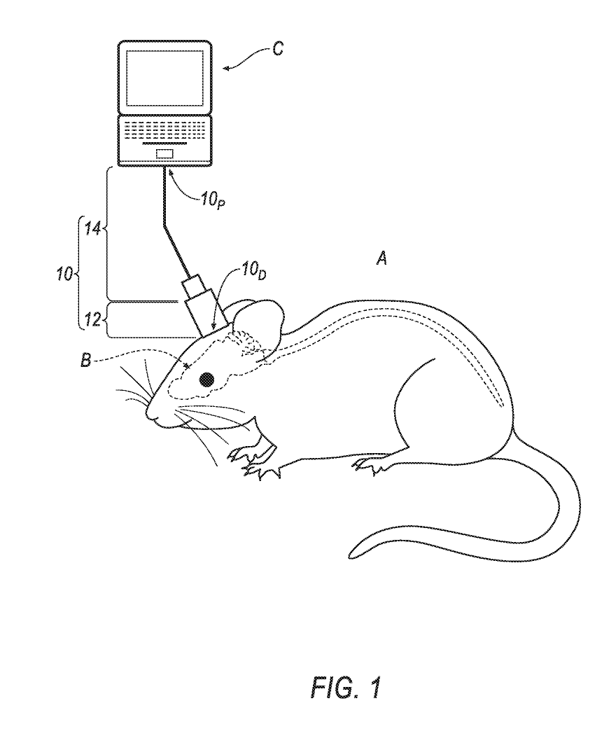

Referring to FIG. 1, an exemplary brain-computer interface (BCI) is shown generally at 10. A proximal end 10_{P} of the BCI 10 is sized for connection to a computing resource C. A portion of the distal end 10_{D} of the BCI 10 is sized for connection to a selected region of a brain B of animalia A.

The computing resource C may be, for example, a digital computer, and may include, but is not limited to one or more electronic digital processors or central processing units (CPUs) in communication with one or more storage resources (e.g., memory, flash memory, dynamic random access memory (DRAM), phase change memory (PCM), and/or disk drives having spindles)). The term “animalia” A may be defined to include any animal species, including but not limited to mice, humans and the like; therefore, although a mouse is illustrated in FIG. 1 as an animal species of animalia, the BCI 10 is not limited for engagement with a mouse and, as a result, the BCI 10 may be sized for engagement with any species including, but not limited to humans as well.

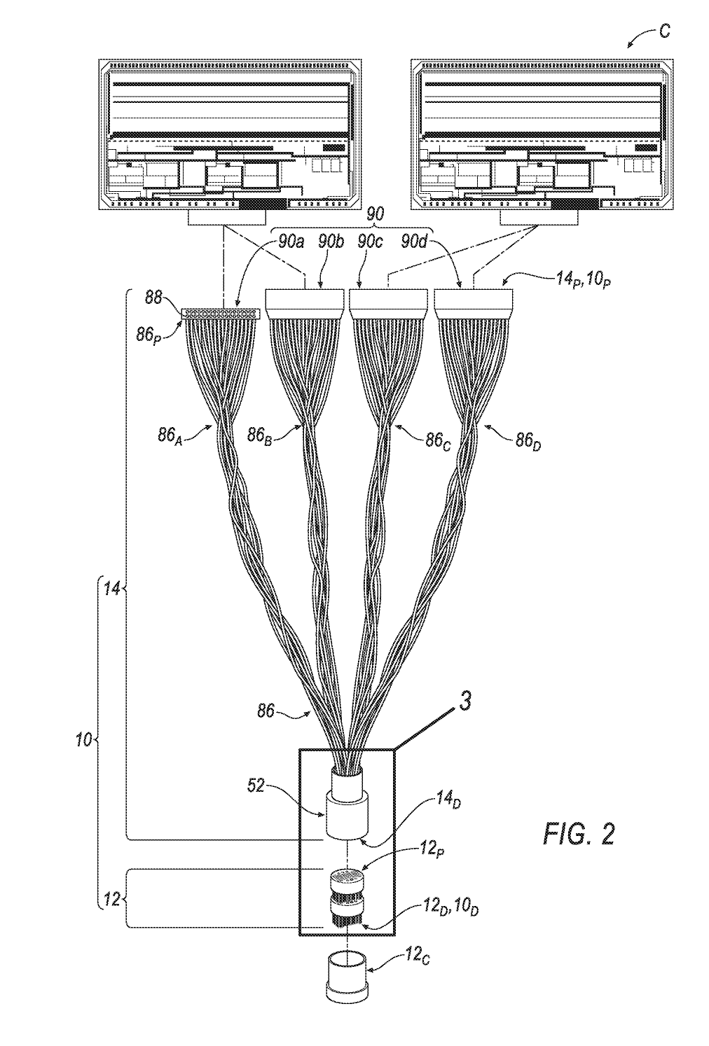

Referring to FIGS. 1 and 2, the BCI 10 generally includes a spatially-adjustable animalia-engaging portion 12 and a computing resource interface portion 14. As seen in FIG. 2, the spatially-adjustable animalia-engaging portion 12 includes a distal end 12_{D} and a proximal end 12_{P}. An outer covering or sleeve 12_{C}, which may be formed from a titanium material, may be arranged over the spatially-adjustable animalia-engaging portion 12 for protecting components (see, e.g., 16a, 16b, 28, 46, 48) of the spatially-adjustable animalia-engaging portion 12. The computing resource interface portion 14 includes a distal end 14_{D} and a proximal end 14_{P}.

At least a portion of the distal end 12_{D} of the spatially-adjustable animalia-engaging portion 12 may generally define at least a portion of the distal end 10_{D} of the BCI 10 that is sized for connection to the brain B of the animalia A. The proximal end 12_{P} of the spatially-adjustable animalia-engaging portion 12 is communicatively-connected to the distal end 14_{D} of the computing resource interface portion 14. The proximal end 14_{P} of the computing resource interface portion 14 may generally define the proximal end 10_{P} of the BCI 10 that is sized for being communicatively-connected to the computing resource C.

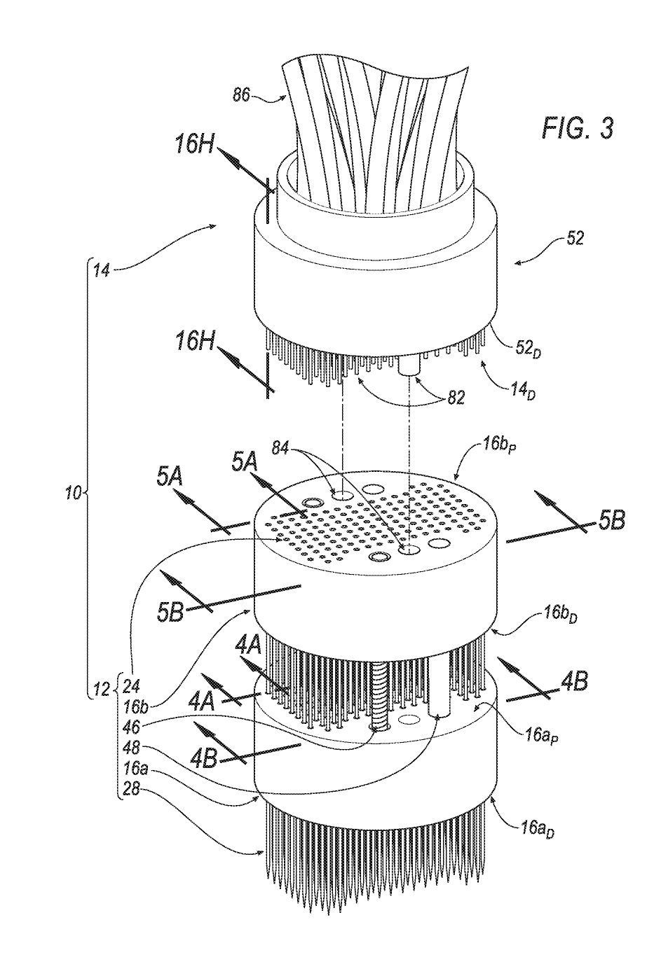

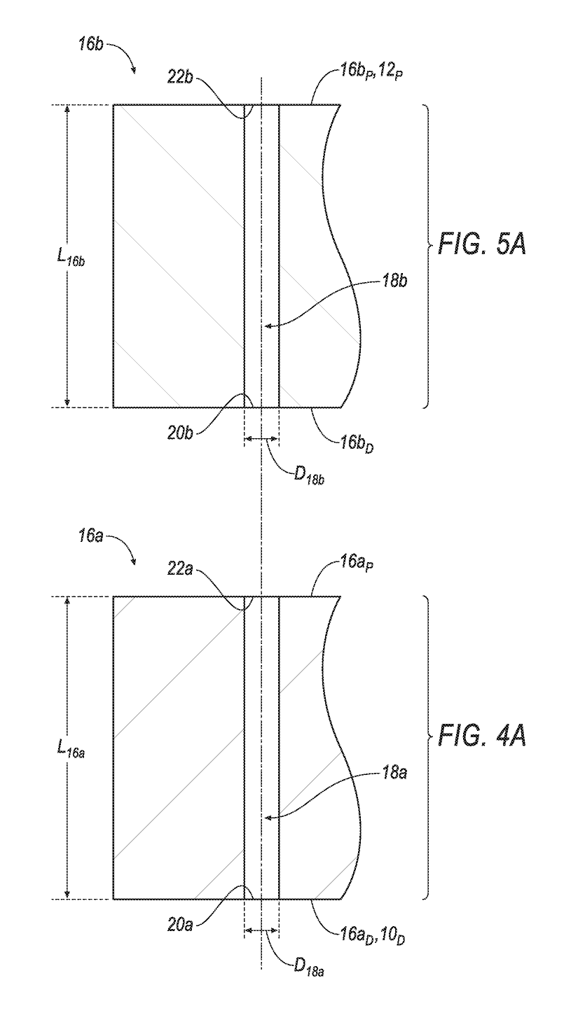

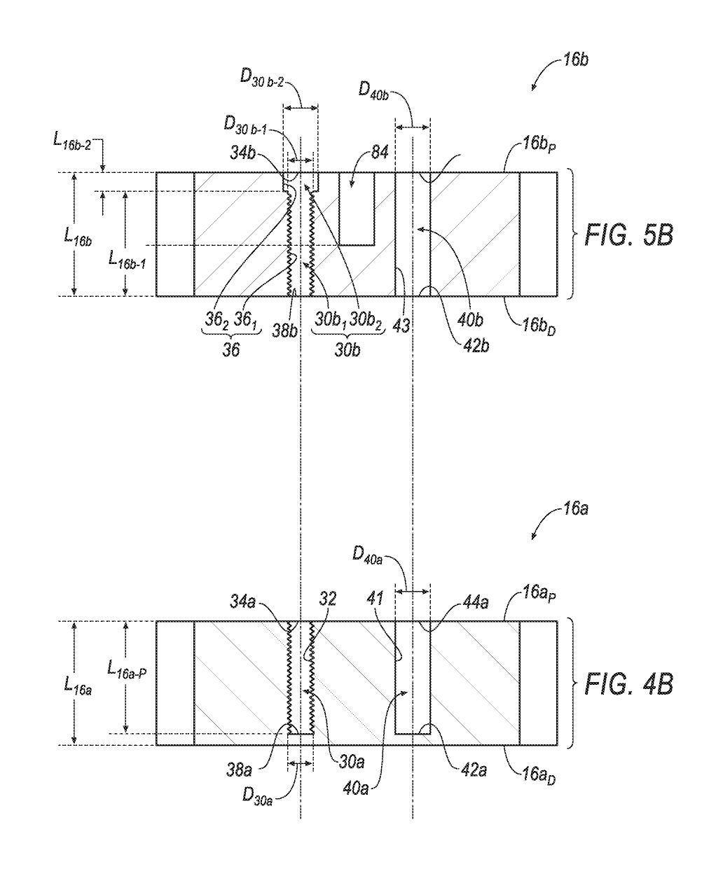

Referring to FIG. 3, the spatially-adjustable animalia-engaging portion 12 includes a (lower) micro-electrode guide portion 16a and a (upper) micro-electrode retainer portion 16b. The micro-electrode guide portion 16a includes a distal end 16a_{D} and a proximal end 16a_{P}. The micro-electrode retainer portion 16b includes a distal end 16b_{D} and a proximal end 16b_{P}. The micro-electrode guide portion 16a is further defined by a length L₁₆ₐ (see, e.g., FIG. 4A) extending between the distal end 16a_{D} and the proximal end 16a_{P}. The micro-electrode retainer portion 16b is also defined by a length L_{16b} (see, e.g., FIG. 5A) extending between the distal end 16b_{D} and the proximal end 16b_{P}.

The distal end 16a_{D} of the micro-electrode guide portion 16a may generally define at least a portion of the distal end 10_{D} of the BCI 10. The proximal end 16a_{P} of the micro-electrode guide portion 16a is arranged in an opposing relationship with respect to the distal end 16b_{D} of the micro-electrode retainer portion 16b. The proximal end 16b of the micro-electrode retainer portion 16b may generally define a portion of the proximal end 12_{P} of the spatially-adjustable animalia-engaging portion 12 and is communicatively-connected to the distal end 14_{D} of the computing resource interface portion 14.

Referring to FIG. 4A, at least one micro-electrode-containing passage 18a extends through the length L₁₆ₐ of the micro-electrode guide portion 16a between the distal end 16a_{D} of the micro-electrode guide portion 16a and the proximal end 16a_{P} of the micro-electrode guide portion 16a. The at least one micro-electrode-containing passage 18a is defined by a diameter D₁₈ₐ. Access to the at least one micro-electrode-containing passage 18a is permitted by a distal opening 20a formed by the distal end 16a_{D} of the micro-electrode guide portion 16a or a proximal opening 22a formed by the proximal end 16a_{P} of the micro-electrode guide portion 16a. In an example, the micro-electrode guide portion 16a defines a plurality of micro-electrode-containing passages 18a. Furthermore, the plurality of micro-electrode-containing passages 18a may be sub-divided or grouped into a plurality of arrays of micro-electrode-containing passages 18a. In some implementations, a silicone elastomer (not shown) is disposed within the distal opening 20a formed by the distal end 16a_{D} of the micro-electrode guide portion 16a. The silicone elastomer seals any gaps, passages or openings between an outer surface 28_{O} of the micro-electrode 28 and the and the inner surface defining the micro-electrode-containing passage 18a, in order to, for example, prevent brain fluid from entering the micro-electrode-containing passage 18a of the micro-electrode guide portion 16a.

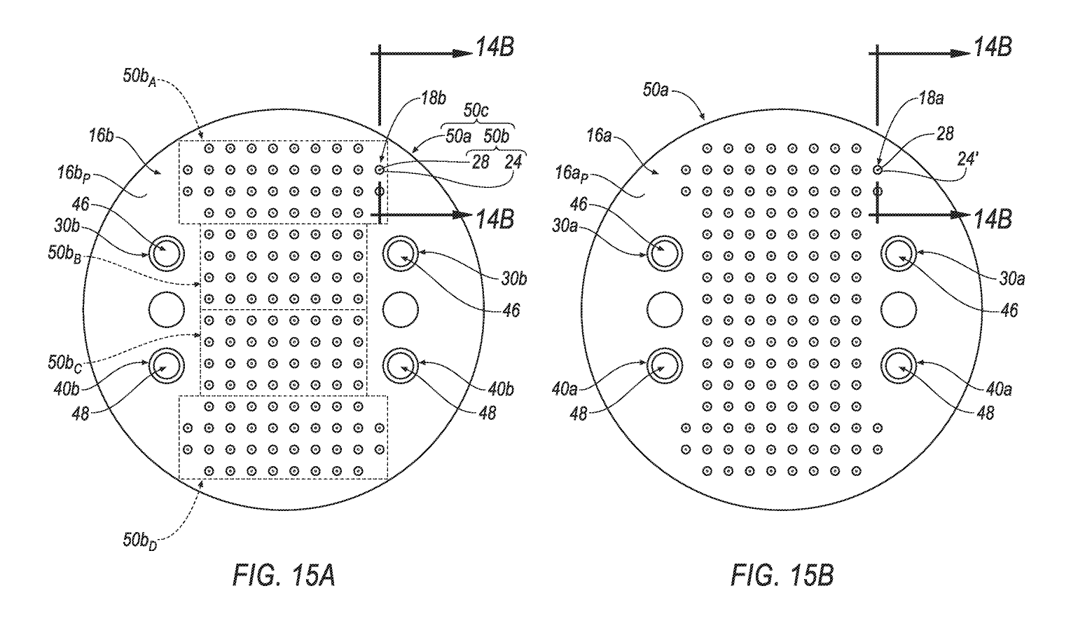

In some examples, the micro-electrode guide portion 16a is formed from a plastic material. The length L₁₆ₐ extending between the distal end 16a_{D} and the proximal end 1ab_{P} of the micro-electrode guide portion 16a may be approximately equal to 3 mm. Each micro-electrode-containing passage 18a may be formed by a drilling process and arranged in a column and row relationship (see, e.g., FIG. 15B) and spaced apart from one another at a distance approximately equal to 0.4 mm.

Referring to FIG. 5A, at least one tube-and-micro-electrode-containing passage 18b extends through the length L_{16b} of the micro-electrode retainer portion 16b between the distal end 16b_{D} of the micro-electrode retainer portion 16b and the proximal end 16b_{P} of the micro-electrode retainer portion 16b. The at least one tube-and-micro-electrode-containing passage 18b is defined by a passage diameter D_{18b}. Access to the at least one tube-and-micro-electrode-containing passage 18b is permitted by a distal opening 20b formed by the distal end 16b_{D} of the micro-electrode retainer portion 16b or a proximal opening 22b formed by the proximal end 16b_{P} of the micro-electrode retainer portion 16b. In an example, the micro-electrode retainer portion 16b defines a plurality of tube-and-micro-electrode-containing passages 18b. Furthermore, the plurality of tube-and-micro-electrode-containing passages 18b may be sub-divided or grouped into a plurality of arrays of tube-and-micro-electrode-containing passages 18b.

In some examples, the micro-electrode retainer portion 16b is formed from a plastic material. The length L_{16b} extending between the distal end 16b_{D} and the proximal end 16b of the micro-electrode retainer portion 16b may be approximately equal to 3 mm. Each passage (see, e.g., 18b in FIG. 5A and 30b, 40b in FIG. 5B) extending there-through may be formed by a drilling process. Each tube-and-micro-electrode-containing passages 18b may be arranged in a column and row relationship (see, e.g., FIG. 15A) and spaced apart from one another at a distance approximately equal to 0.4 mm.

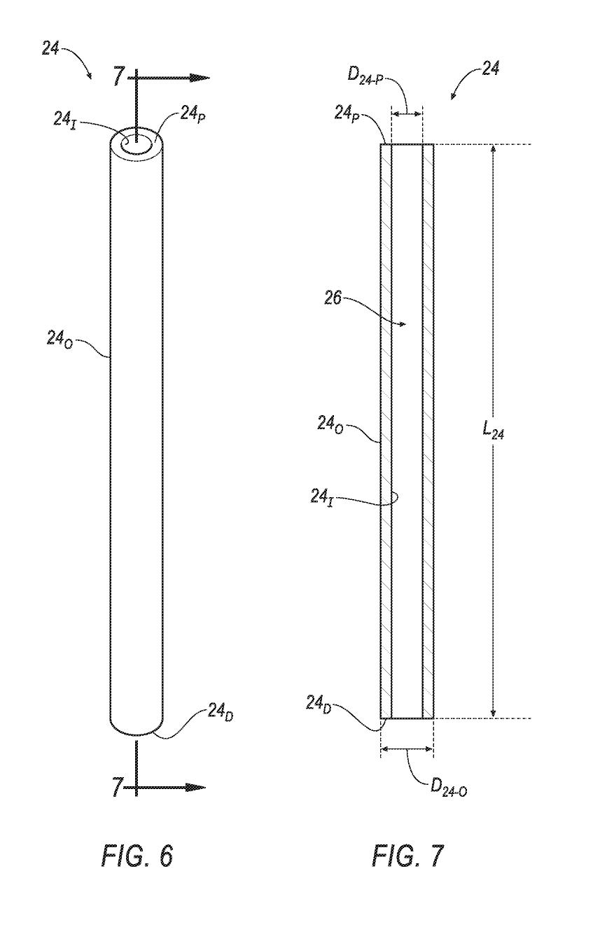

Referring to FIGS. 6 and 7, the spatially-adjustable animalia-engaging portion 12 further includes at least one micro-electrode-containing tube 24, which may be formed from stainless steel. The at least one micro-electrode-containing tube 24 is defined by a distal surface 24_{D}, a proximal surface 24_{P}, an outer surface 24_{O} and an inner surface 24_{I}. The at least one micro-electrode-containing tube 24 is further defined by a length L₂₄ (see, e.g., FIG. 7) extending between the distal surface 24_{D} and the proximal surface 24_{P}. Referring to FIG. 7, the inner surface 24_{I} of the micro-electrode-containing tube 24 further defines a micro-electrode-receiving passage 26 extending through the length L₂₄ of the micro-electrode-containing tube 24 from the distal surface 24_{D} to the proximal surface 24_{P}. Yet even further, the outer surface 24_{O} of the micro-electrode-containing tube 24 defines an outer diameter D_{24-O} of the micro-electrode-containing tube 24. Furthermore, the inner surface 24_{I} of the micro-electrode-containing tube 24 defines a passage diameter D_{24-P} of the micro-electrode-containing tube 24.

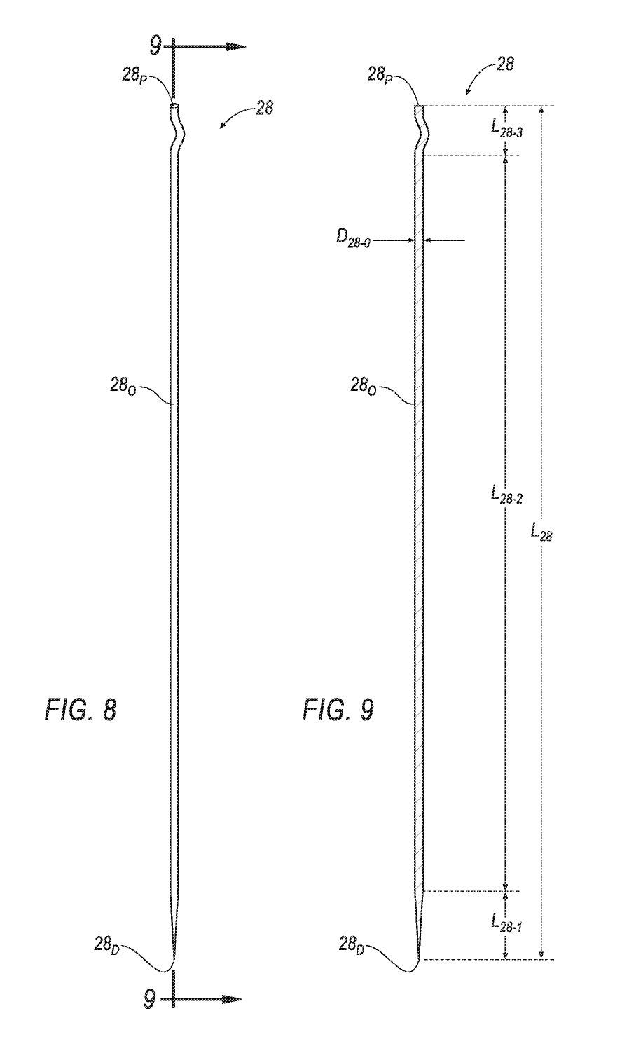

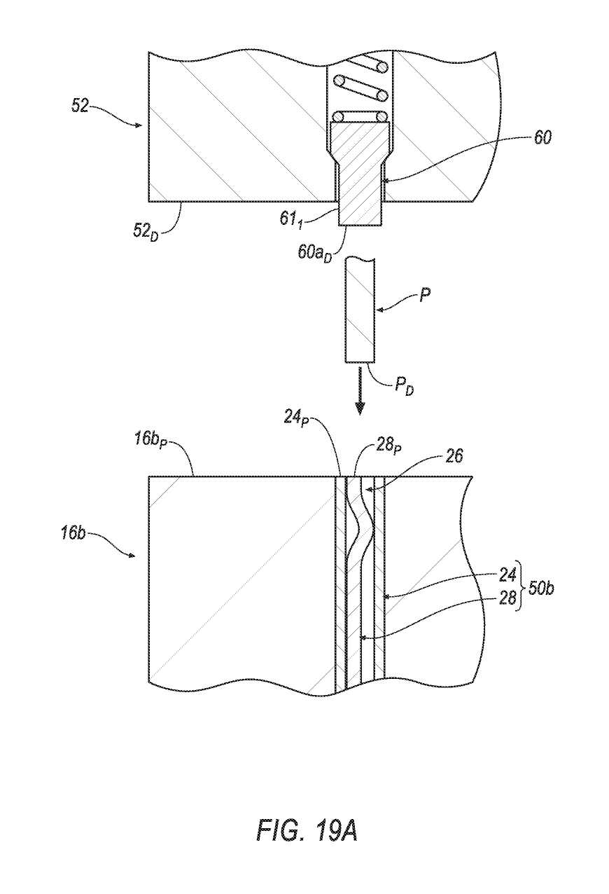

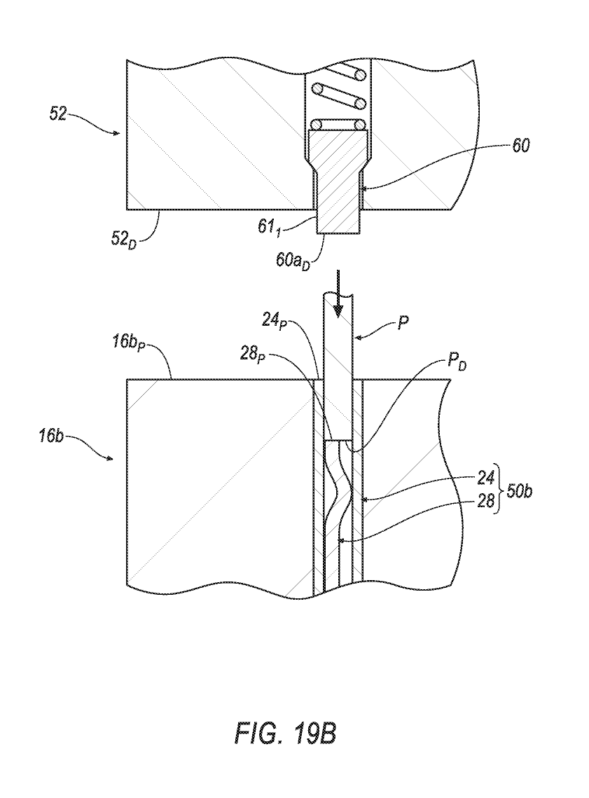

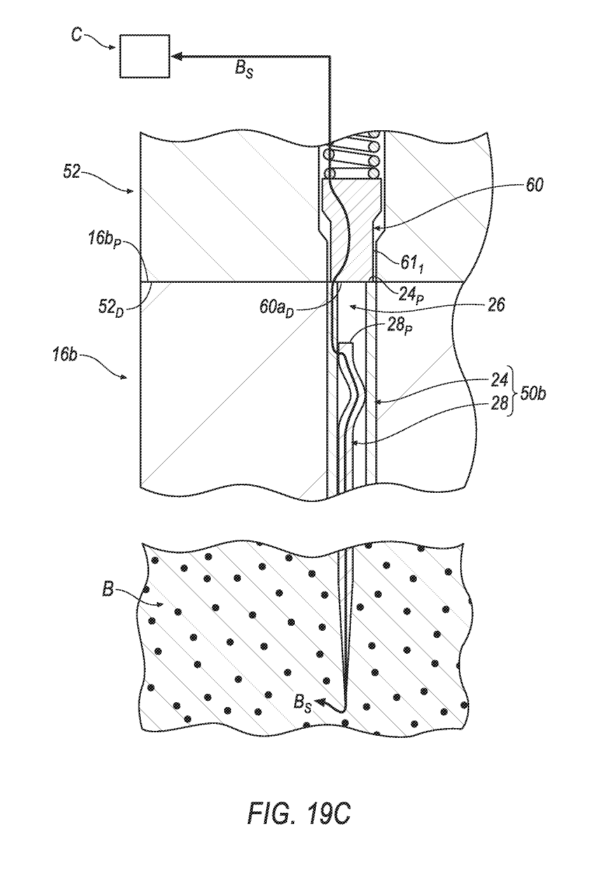

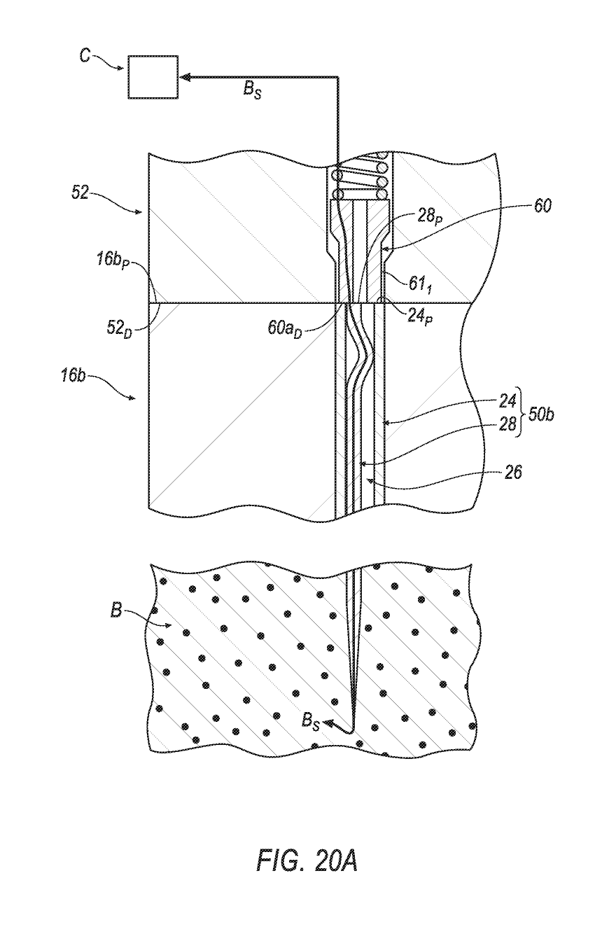

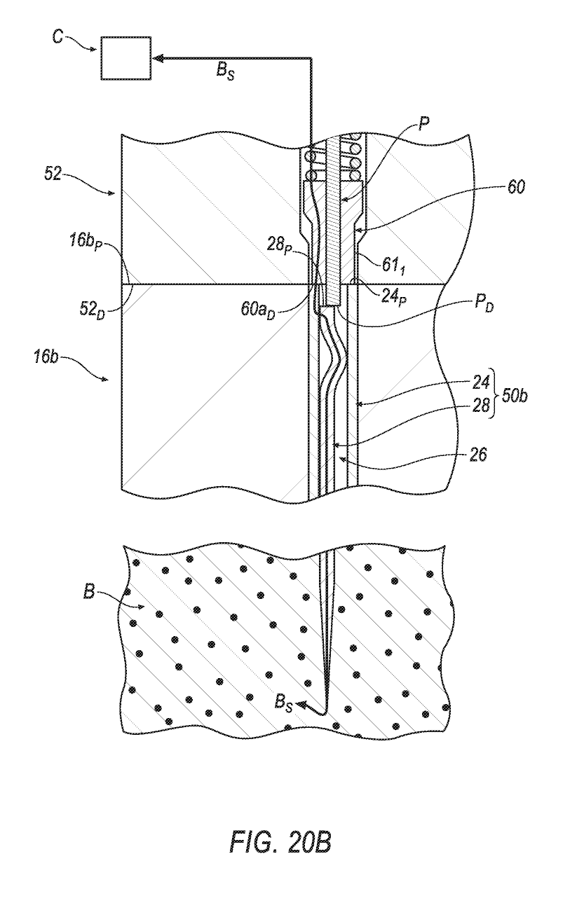

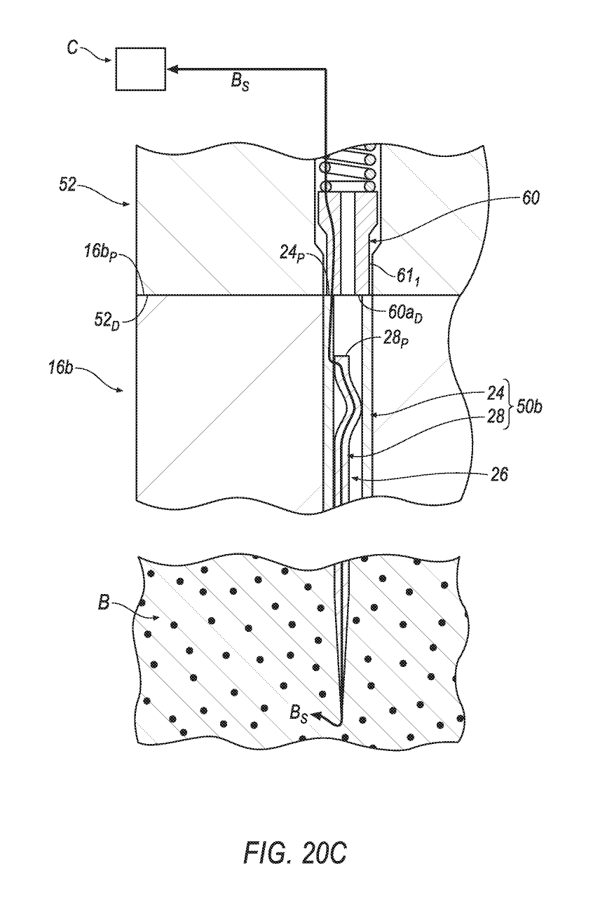

Referring to FIGS. 8 and 9, the spatially-adjustable animalia-engaging portion 12 further includes at least one micro-electrode 28. The at least one micro-electrode 28 is defined by a distal tip 28_{D}, a proximal surface 28_{P} and an outer surface 28_{O}. The at least one micro-electrode 28 is further defined by a length L₂₈ (see, e.g., FIG. 9) extending between the distal tip 28_{D} and the proximal surface 28_{P}. Referring to FIG. 9, the outer surface 28_{O} of the micro-electrode 28 defines an outer diameter D_{28-O} of the micro-electrode 28. The distal tip 28_{D} of the at least one micro-electrode 28 may be defined by a sharp or pointed tip to facilitate piercing of an outer surface of the brain B of the animalia A as the at least one micro-electrode 28 is inserted into the brain B of the animalia A. The proximal surface 28_{P} of the at least one micro-electrode 28 may be by a substantially flat surface to facilitate engagement of the at least one micro-electrode 28 with a pushing device (see, e.g., push-pin P in FIGS. 19A-19C). In an example, the at least one micro-electrode 28 may be defined by a plurality of micro-electrodes 28. Furthermore, the plurality of micro-electrodes 28 may be sub-divided or grouped into a plurality of arrays of micro-electrodes 28.

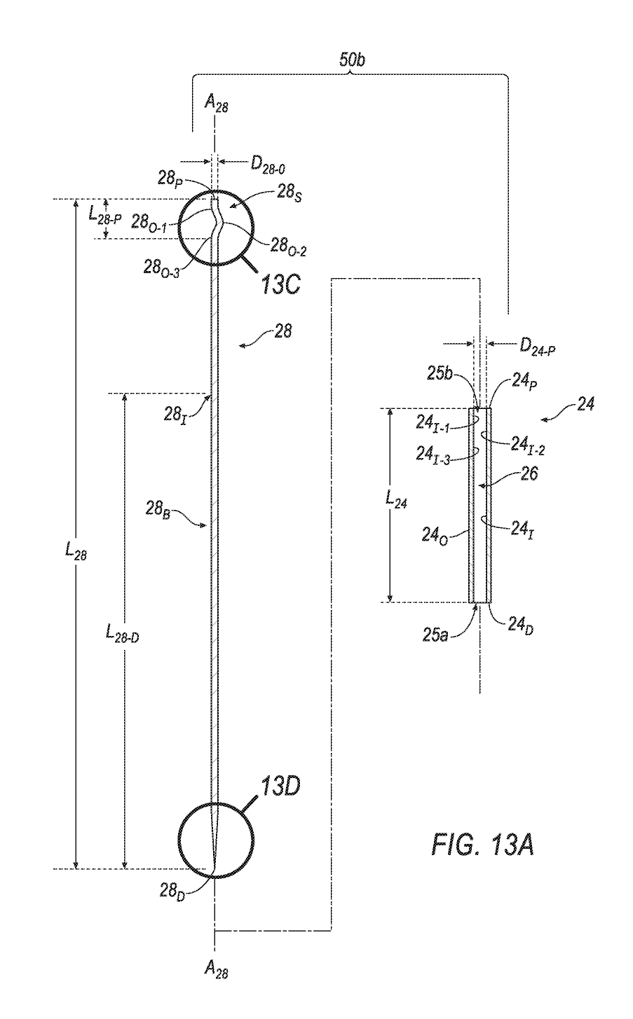

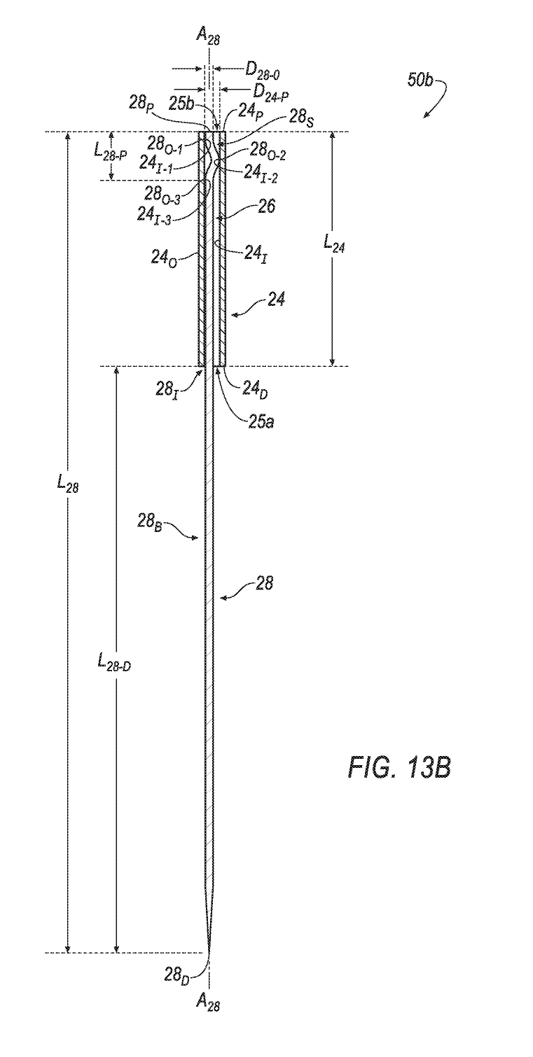

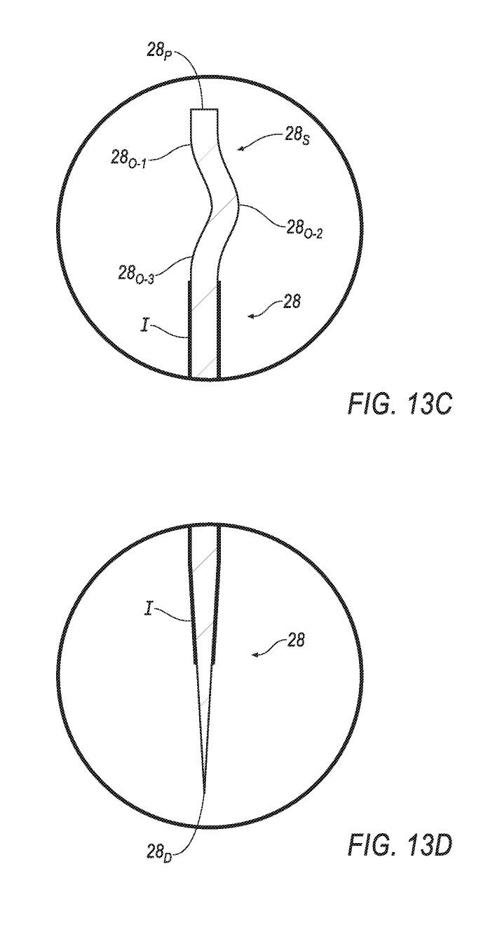

As will be described with reference to FIGS. 13A and 13B, the micro-electrode 28 may be disposed within the micro-electrode-containing tube 24 for forming a “second subassembly” 50b. The second subassembly 50b may be interfaced with the micro-electrode-containing passage 18a and the tube-and-micro-electrode-containing passage 18b described above at FIGS. 4A and 5A.

In some implementations, the micro-electrodes 28 is formed from conductive filaments (e.g., micro-wires) including, for example, one or more of a combination of metal and/or non-metal materials including, but not limited to stainless steel wires, tungsten wires, platinum, iridium wires, pure iridium wires, carbon fibers and conductive polymers. Furthermore, an insulator I (see, e.g., FIGS. 13C-13D), such as, non-conductive polymer coating (e.g., polyamide) may be disposed over a portion of the micro-electrodes 28.

In some examples, platinum/iridium wires are comparatively stiffer with respect to stainless steel wires and are more resistant to erosion when compared to stainless steel wires and tungsten wires, which may therefore result in a more favorable material selection if the micro-electrodes 28 are chronically implanted and repeatedly stimulated with signals B_{S}. For example, the micro-electrodes 28 may be formed from a platinum/iridium alloy having an outer diameter D_{28-O} approximately equal to 12.5 μm, 25 μm or 50 μm and a length L₂₈ ranging between approximately 10 mm-40 mm.

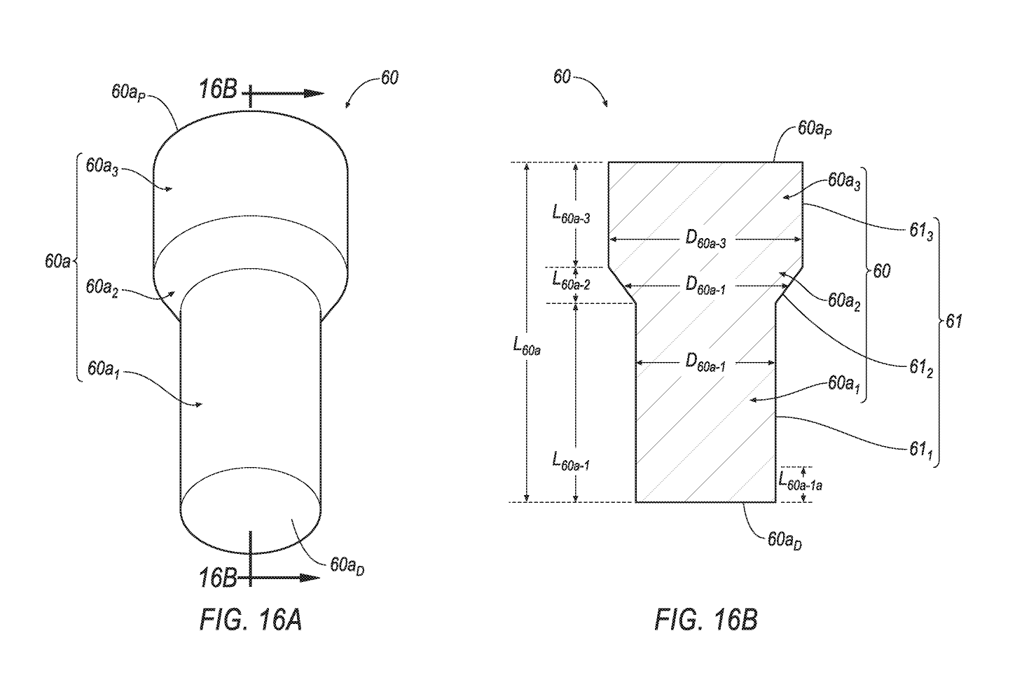

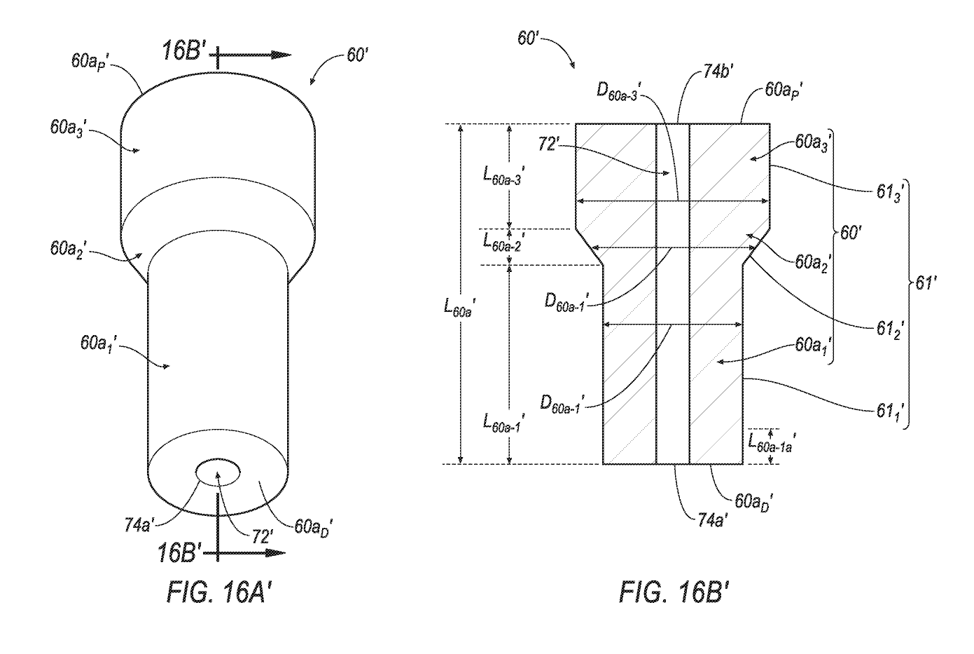

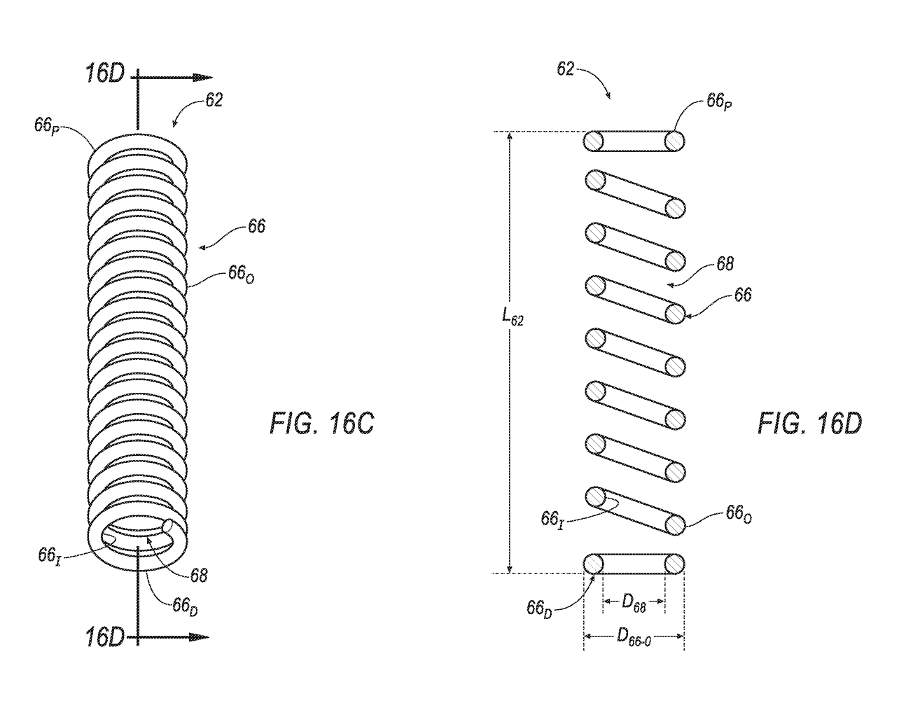

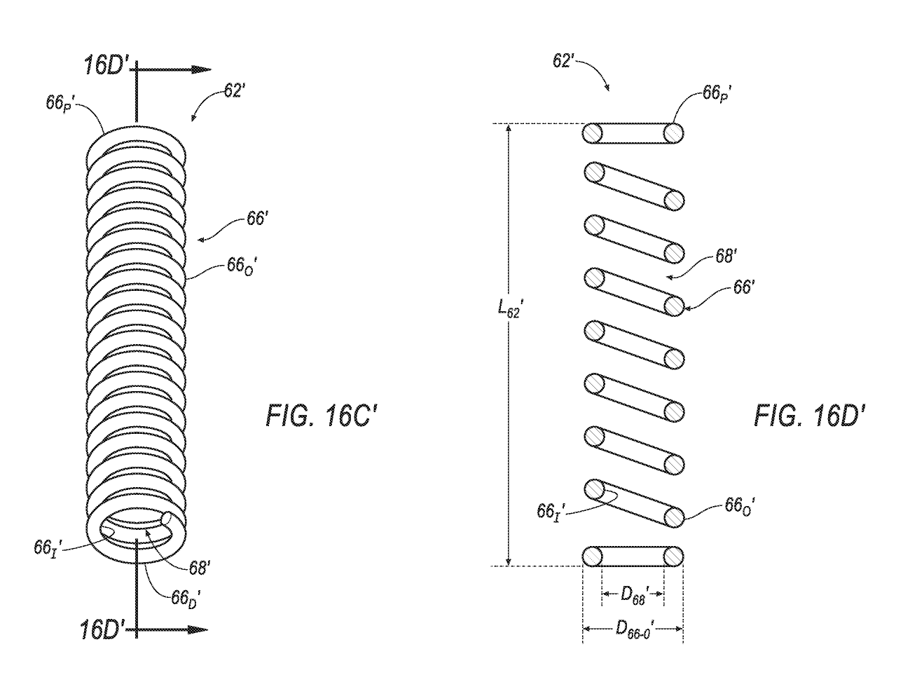

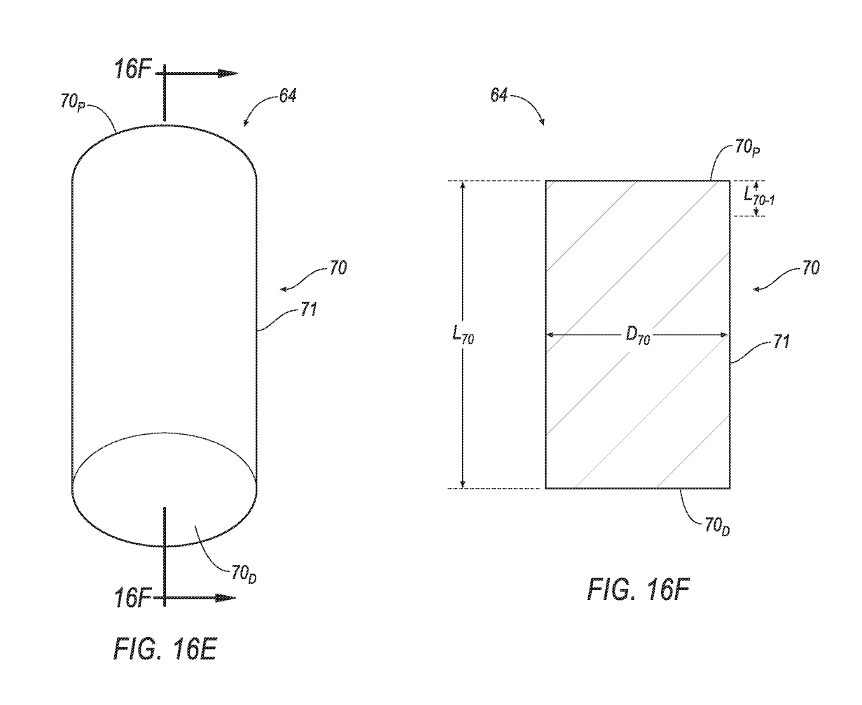

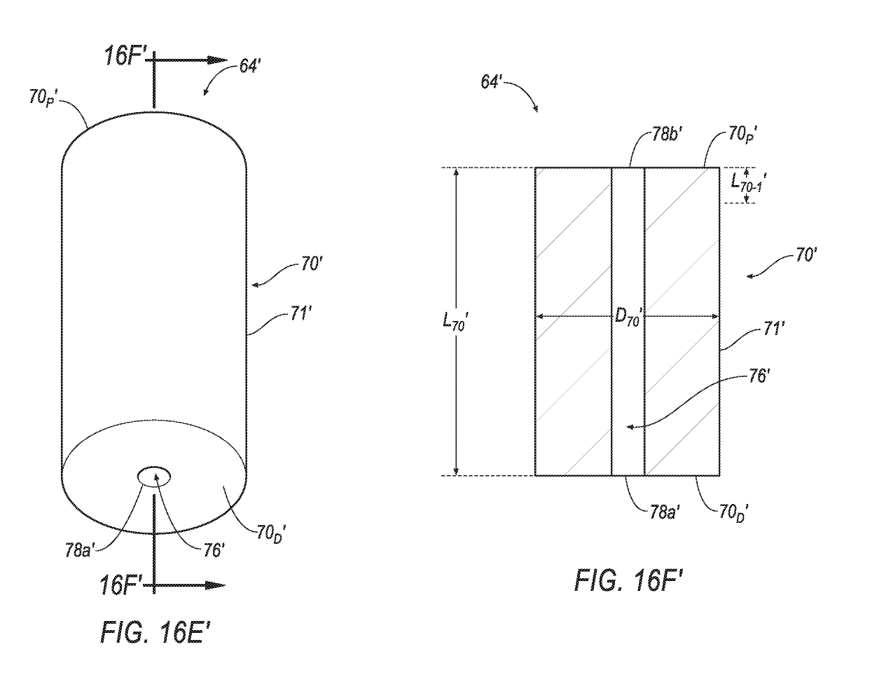

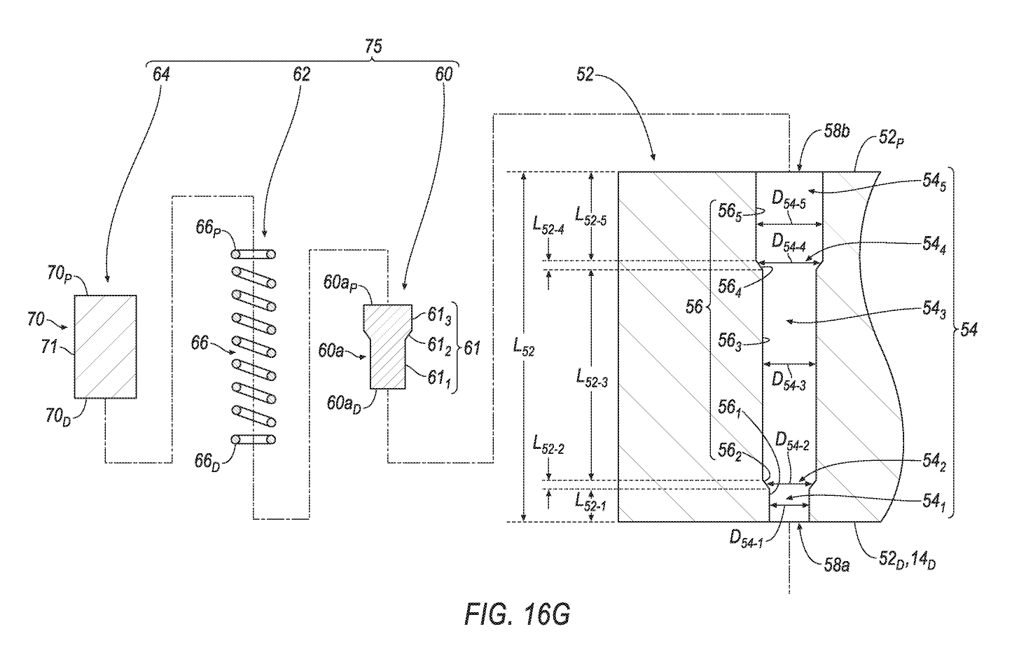

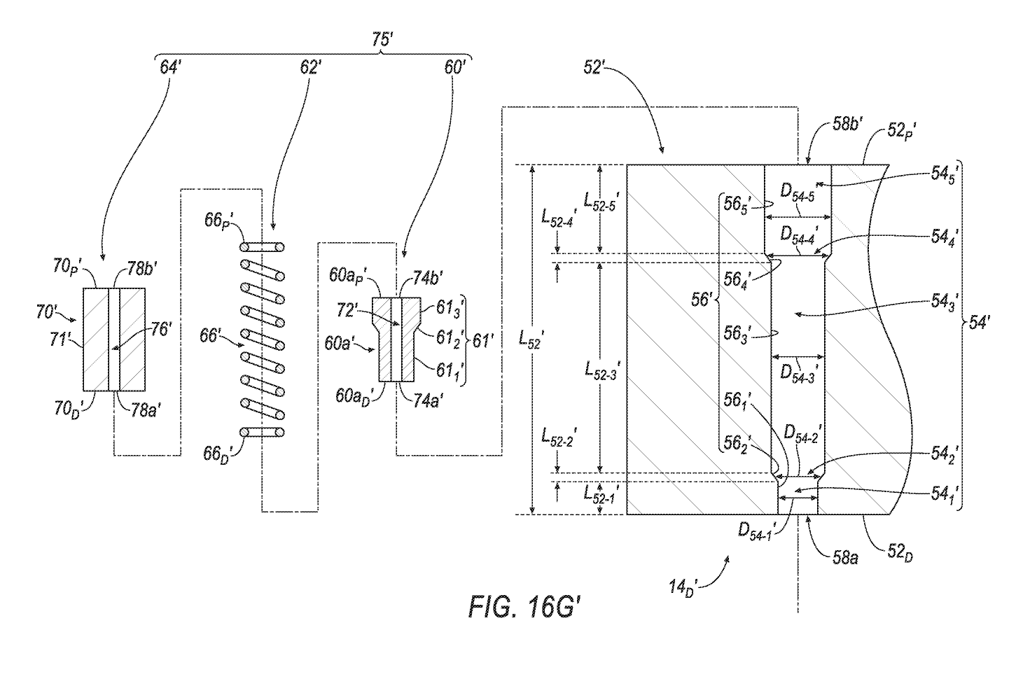

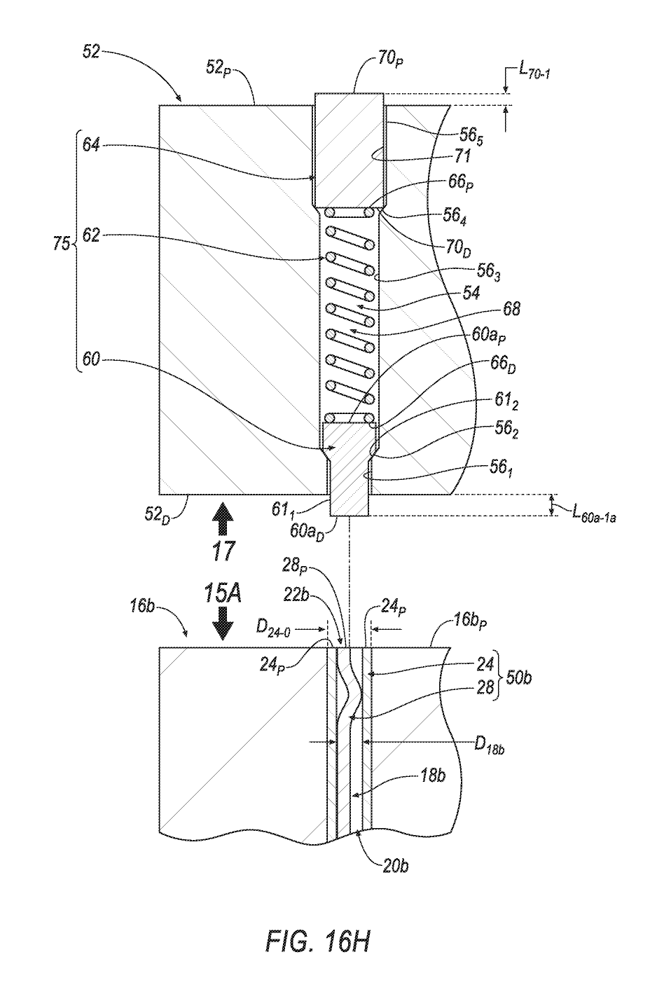

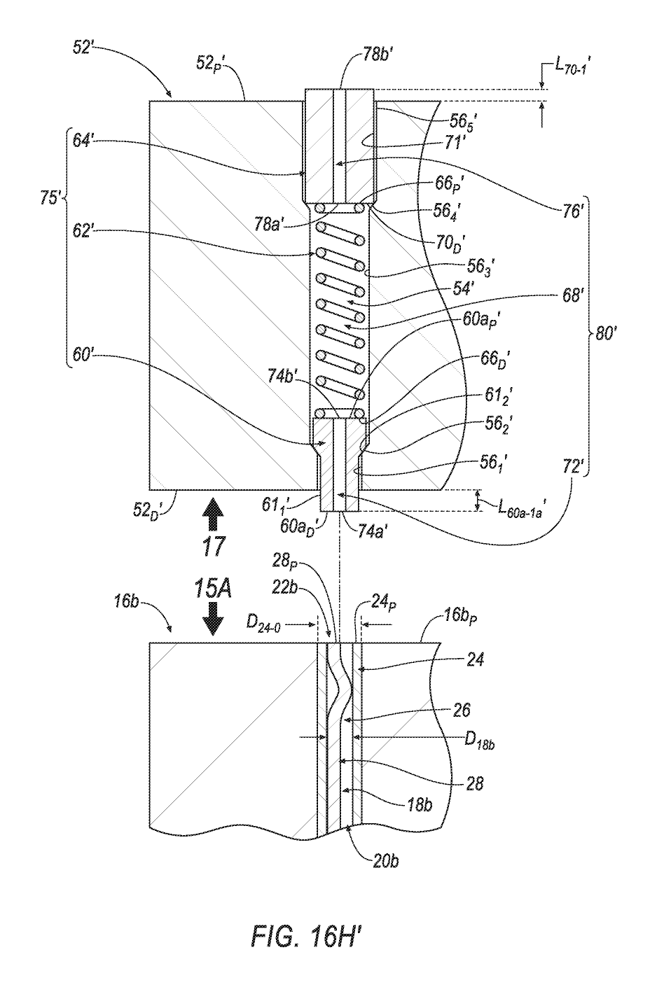

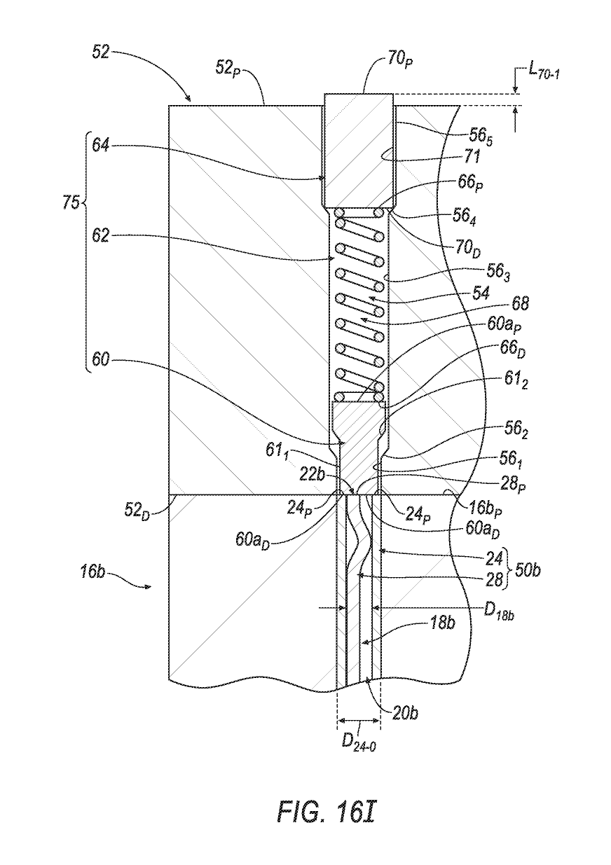

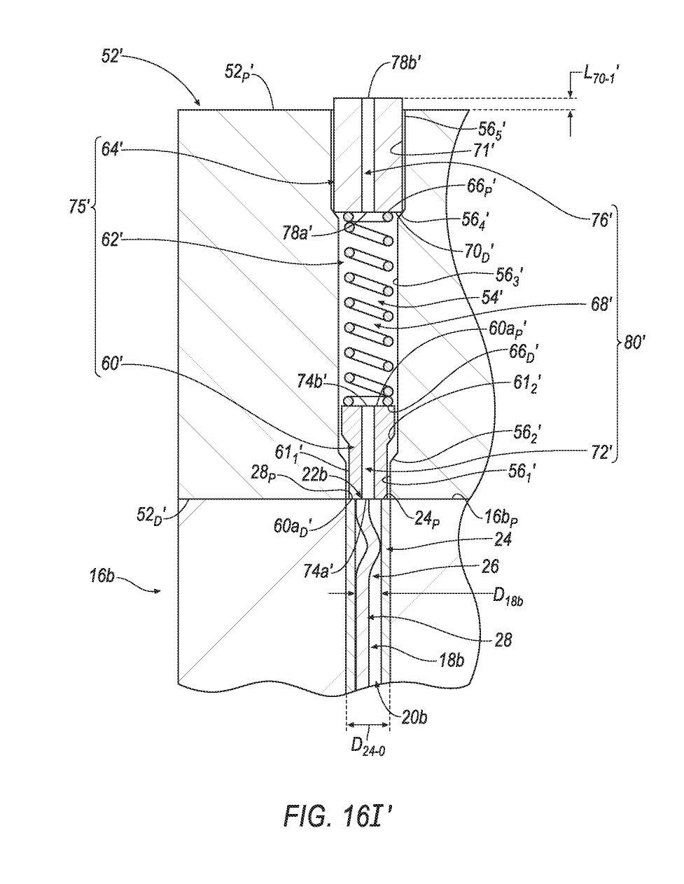

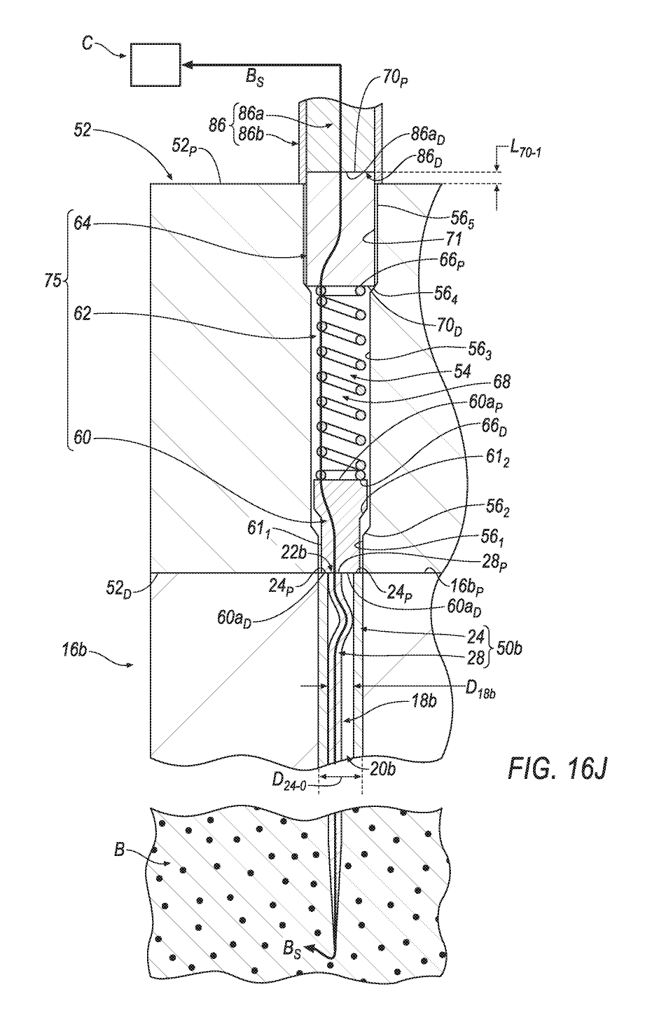

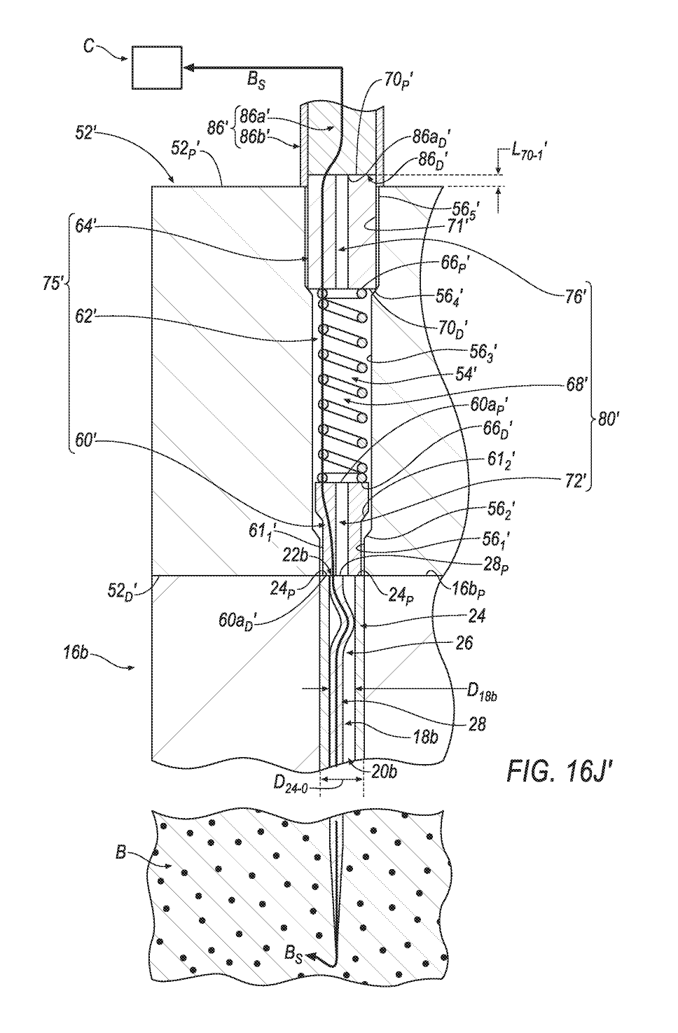

Furthermore, the distal tip 28_{D} of each micro-electrode 28 may be sharpened by using a chemical etching lesion process or an abrasive grinding wheel (e.g., a diamond grinder) in order to, for example, increase a signal/noise ratio of signals (see, e.g., Bs in FIGS. 16J, 16J′, 18A-18B, 19C and 20A-20C) being communicated to/from a brain B and a computing resource C when detecting neuronal activity and local field potentials of the brain B. In other examples, electrical sparking, laser cutting, or the like may be utilized for sharpening the distal tip 28_{D} of each micro-electrode 28.

Referring to FIG. 4B, at least one spatial-adjuster-component-containing passage 30a partially extends through a portion L_{16a-P} of the length L₁₆ₐ of the micro-electrode guide portion 16a from the proximal end 16a_{P} of the micro-electrode guide portion 16a and toward (but not all the way to) the distal end 16a_{D} of the micro-electrode guide portion 16a. The at least one spatial-adjuster-component-containing passage 30a is defined by an inner surface 32 defining a spatial-adjuster-component-containing passage diameter D₃₀ₐ. Furthermore, the inner surface 32 may define a threaded surface. Access to the at least one spatial-adjuster-component-containing passage 30a is permitted by a proximal opening 34a formed by the proximal end 16a_{P} of the micro-electrode guide portion 16a. Because the at least one spatial-adjuster-component-containing passage 30a does not extend through the entire length L₁₆ₐ of the micro-electrode guide portion 16a, access to the at least one spatial-adjuster-component-containing passage 30a from the distal end 16a_{D} of the micro-electrode guide portion 16a is not permitted. Therefore, the at least one spatial-adjuster-component-containing passage 30a may be further defined by a distal end wall surface 38a connected to the inner surface 32 (i.e., the portion L_{16a-P} of the length L₁₆ₐ extends between the proximal end 16a_{P} and the distal end wall surface 38a). The micro-electrode guide portion 16a defines a pair of spatial-adjuster-component-containing passages 30a including a first spatial-adjuster-component-containing passage and a second spatial-adjuster-component-containing passage.

Referring to FIG. 5B, at least one spatial-adjuster-component-containing passage 30b extends through all of the length L_{16b} of the micro-electrode retainer portion 16b between the distal end 16b_{D} of the micro-electrode retainer portion 16b and the proximal end 16b_{P} of the micro-electrode retainer portion 16b. The at least one spatial-adjuster-component-containing passage 30b is defined by: (1) a first portion 36₁ of an inner surface 36 that defines a first spatial-adjuster-component-containing passage diameter D_{30b-1} of the at least one spatial-adjuster-component-containing passage 30b; and (2) a second portion 36₂ of the inner surface 36 that defines a second spatial-adjuster-component-containing passage diameter D_{30b-2} of the at least one spatial-adjuster-component-containing passage 30b.

A first portion 30b₁ of the at least one spatial-adjuster-component-containing passage 30b defined by the first spatial-adjuster-component-containing passage diameter D_{30b-1} extends along a first portion L_{16b-1} of the length L_{16b} from the distal end 16b_{D} of the micro-electrode retainer portion 16b. A second portion 30b2 of the at least one spatial-adjuster-component-containing passage 30b defined by the second spatial-adjuster-component-containing passage diameter D_{30b-2} extends along a second portion L_{16b-2} of the length L_{16b} from the proximal end 16b_{P} of the micro-electrode retainer portion 16b. The second spatial-adjuster-component-containing passage diameter D_{30b-2} is greater than the first spatial-adjuster-component-containing passage diameter D_{30b-1}.

The first portion 36₁ of the inner surface 36 may define a non-threaded surface and the second portion 36₂ of the inner surface 36 may define a non-threaded surface. Access to the at least one spatial-adjuster-component-containing passage 30b is permitted by a distal opening 38b formed by the distal end 16b_{D} of the micro-electrode retainer portion 16b or a proximal opening 34b formed by the proximal end 16b_{P} of the micro-electrode retainer portion 16b. The micro-electrode retainer portion 16b defines a pair of spatial-adjuster-component-containing passages including a first spatial-adjuster-component-containing passage and a second spatial-adjuster-component-containing passage.

Referring to FIG. 4B, at least one spatial-adjuster-guide-post-containing passage 40a partially extends through a portion (that may be substantially similar to the portion L_{16a-P}) of the length L₁₆ₐ of the micro-electrode guide portion 16a from the proximal end 16a_{P} of the micro-electrode guide portion 16a and toward (but all the way to) the distal end 16a_{D} of the micro-electrode guide portion 16a. The at least one spatial-adjuster-guide-post passage 40a is defined by an inner surface 41 defining a spatial-adjuster-guide-post-containing passage diameter D₄₀ₐ. Access to the at least one spatial-adjuster-guide-post passage 40a is permitted by a proximal opening 44a formed by the proximal end 16a_{P} of the micro-electrode guide portion 16a. Because the at least one spatial-adjuster-guide-post-containing passage 40a does not extend through the entire length L₁₆ₐ of the micro-electrode guide portion 16a, access to the at least one spatial-adjuster-guide-post-containing passage 40a from the distal end 16a_{D} of the micro-electrode guide portion 16a is not permitted. Therefore, the at least one spatial-adjuster-guide-post-containing passage 40a may be further defined by a distal end wall surface 42a connected to the inner surface 41 (i.e., the portion L_{16a-P} of the length L₁₆ₐ may extend between the distal end 16a_{D} and the distal end wall surface 42a). Furthermore, both of the inner surface 41 and the distal end wall surface 42a may be defined by a smooth, non-threaded surface. The micro-electrode guide portion 16a defines a pair of spatial-adjuster-guide-post passages including a first spatial-adjuster-guide-post passage and a second spatial-adjuster-component-containing passage.

Referring to FIG. 5B, at least one spatial-adjuster-guide-post passage 40b extends through all of the length L_{16b} of the micro-electrode retainer portion 16b between the distal end 16b_{D} of the micro-electrode retainer portion 16b and the proximal end 16b_{P} of the micro-electrode retainer portion 16b. The at least one spatial-adjuster-guide-post passage 40b is defined by an inner surface 43 defining a spatial-adjuster-guide-post passage diameter D_{40b}. The inner surface 43 may be defined by a smooth, non-threaded surface. Access to the at least one spatial-adjuster-guide-post passage 40b is permitted by a distal opening 42b formed by the distal end 16b_{D} of the micro-electrode retainer portion 16b or a proximal opening 44b formed by the proximal end 16b_{P} of the micro-electrode retainer portion 16b. The micro-electrode retainer portion 16b defines a pair of spatial-adjuster-guide-post passages including a first spatial-adjuster-guide-post passage and a second spatial-adjuster-guide-post-containing passage.

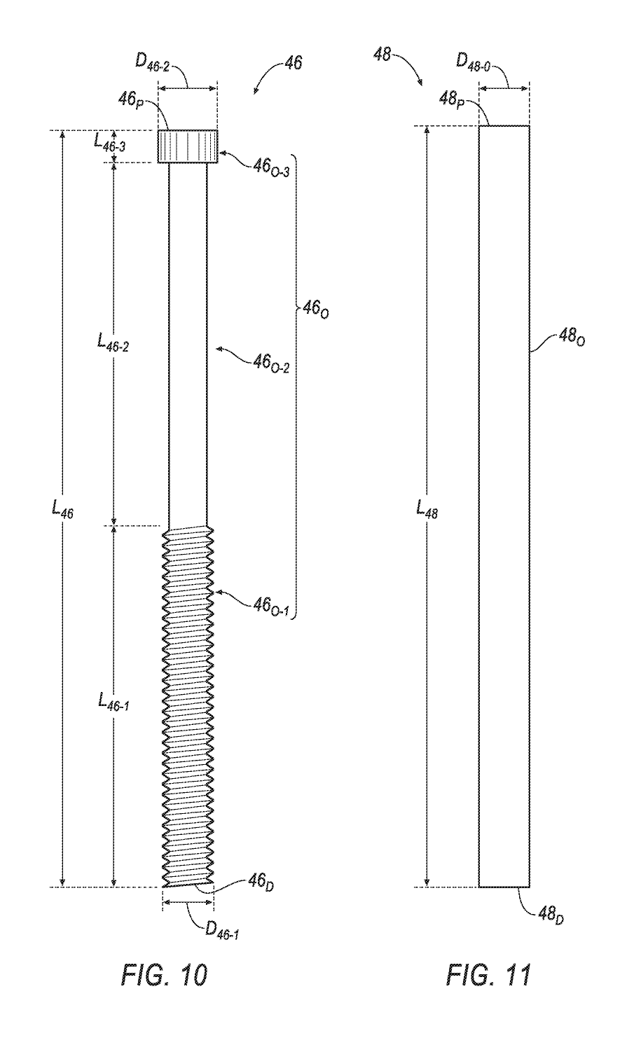

Referring to FIG. 10, the spatially-adjustable animalia-engaging portion 12 further includes at least one spatial-adjuster-component 46, which may be formed from a titanium material. The at least one spatial-adjuster-component 46 is defined by a distal surface 46D, a proximal surface 46_{P} and an outer surface 46_{O}. The at least one spatial-adjuster-component 46 is further defined by a length L₄₆ extending between the distal surface 46D and the proximal surface 46_{P}. The outer surface 46_{O} of the spatial-adjuster-component 46 defines a first outer diameter D₄₆₋₁ of the spatial-adjuster-component 46 and a second outer diameter D₄₆₋₂ of the spatial-adjuster-component 46.

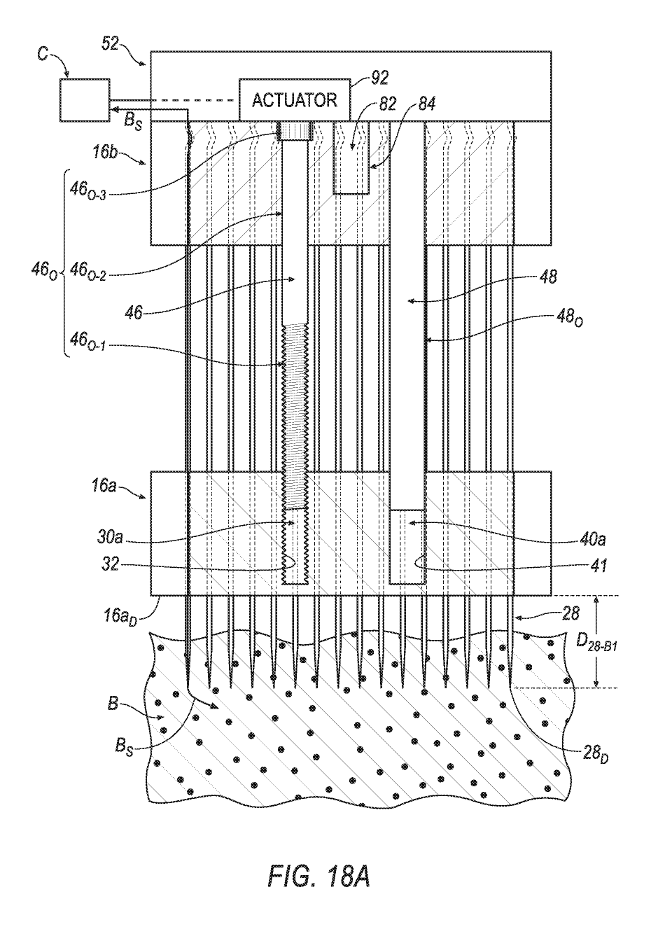

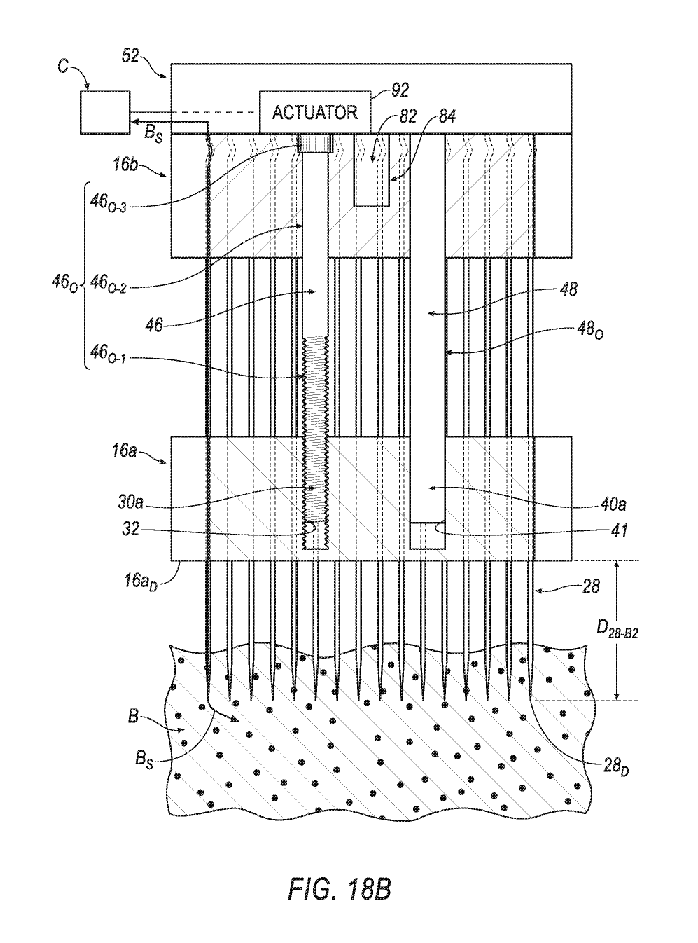

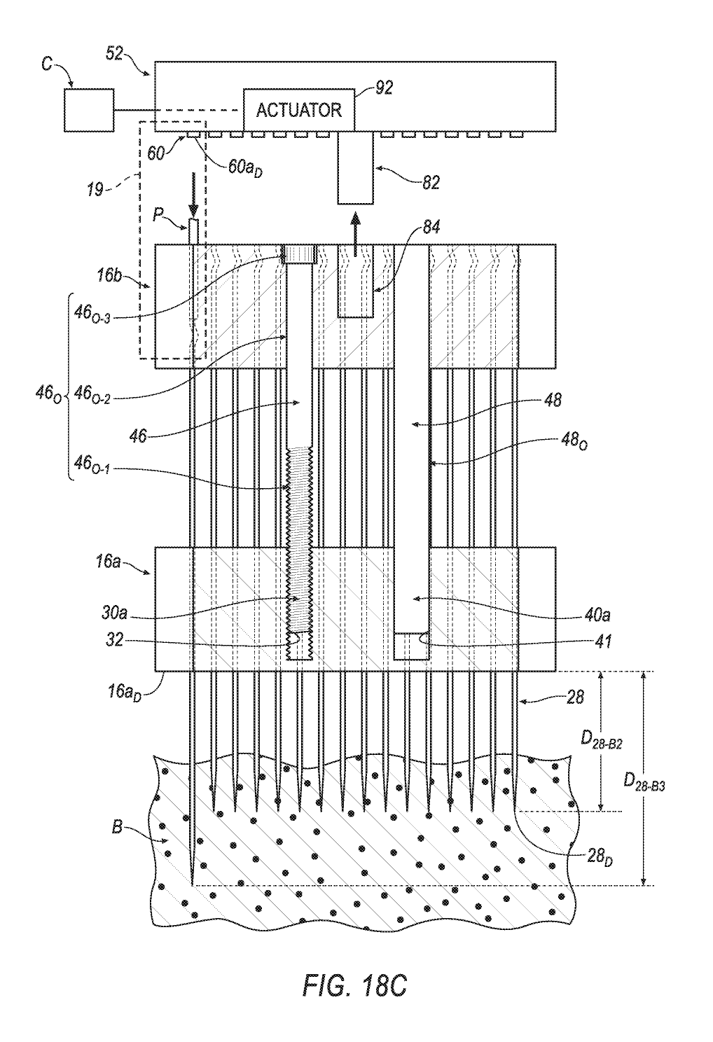

In some examples, the length L₄₆ of the spatial-adjuster-component 46 may be approximately equal to 0.25″ (i.e., ¼ of an inch). In an example, each turn (see, e.g., R in FIG. 18B) may be approximately equal to 1/120 of an inch/0.212 mm.

Further, a first portion L₄₆₋₁ of the length L₄₆ of the spatial-adjuster-component 46 and a second portion L₄₆₋₂ of the length L₄₆ of the spatial-adjuster-component 46 are defined by the first outer diameter D₄₆₋₁. Yet even further, a third portion L₄₆₋₃ of the length L₄₆ of the spatial-adjuster-component 46 is defined by the second outer diameter D₄₆₋₂. The second outer diameter D₄₆₋₂ is greater than the first outer diameter D₄₆₋₁.

Furthermore, a first portion 46_{O-1} of the outer surface 46_{O} defined by the first portion L₄₆₋₁ of the length L₄₆ of the spatial-adjuster-component 46 may be defined by a threaded surface and a second portion 46_{O-2} of the outer surface 46_{O} defined by the second portion L₄₆₋₂ of the length L₄₆ of the spatial-adjuster-component 46 may be defined by a non-threaded surface. In some examples, the non-threaded surface is formed by removing or abrading threads that were previously present on the second portion 46_{O-2} of the outer surface 46_{O}. Yet even further, a third portion 46_{O-3} of the outer surface 46_{O} defined by the third portion L₄₆₋₃ of the length L₄₆ of the spatial-adjuster-component 46 may be defined by an actuator-engagement surface (e.g., for mechanical engagement with an actuator). For example, the third portion 46_{O-3} of the outer surface 46_{O} may include a frictional or keyed configuration that is interfaceable with a corresponding surface configuration of the actuator (see, e.g., actuator 92 in FIGS. 18A-18C that rotates R the at least one spatial-adjuster-component 46 in a clockwise or counter-clockwise direction)). The at least one spatial-adjuster-component 46 may be defined by a pair of spatial-adjuster-components including a first spatial-adjuster-component and a second spatial-adjuster-component.

Referring to FIG. 11, the spatially-adjustable animalia-engaging portion 12 further includes at least one spatial-adjuster-guide-post 48. The at least one spatial-adjuster-guide-post 48 is defined by a distal surface 48D, a proximal surface 48_{P} and an outer surface 48_{O}. The outer surface 48_{O} may be defined by a smooth, non-threaded surface. The at least one spatial-adjuster-guide-post 48 is further defined by a length L₄₈ extending between the distal surface 48D and the proximal surface 48_{P}. The outer surface 48_{O} of the spatial-adjuster-guide-post 48 defines an outer diameter D_{48-O} of the spatial-adjuster-guide-post 48. The at least one spatial-adjuster-guide-post 48 may be defined by a pair of spatial-adjuster-guide-posts including a first spatial-adjuster-guide-post and a second spatial-adjuster-guide-post.

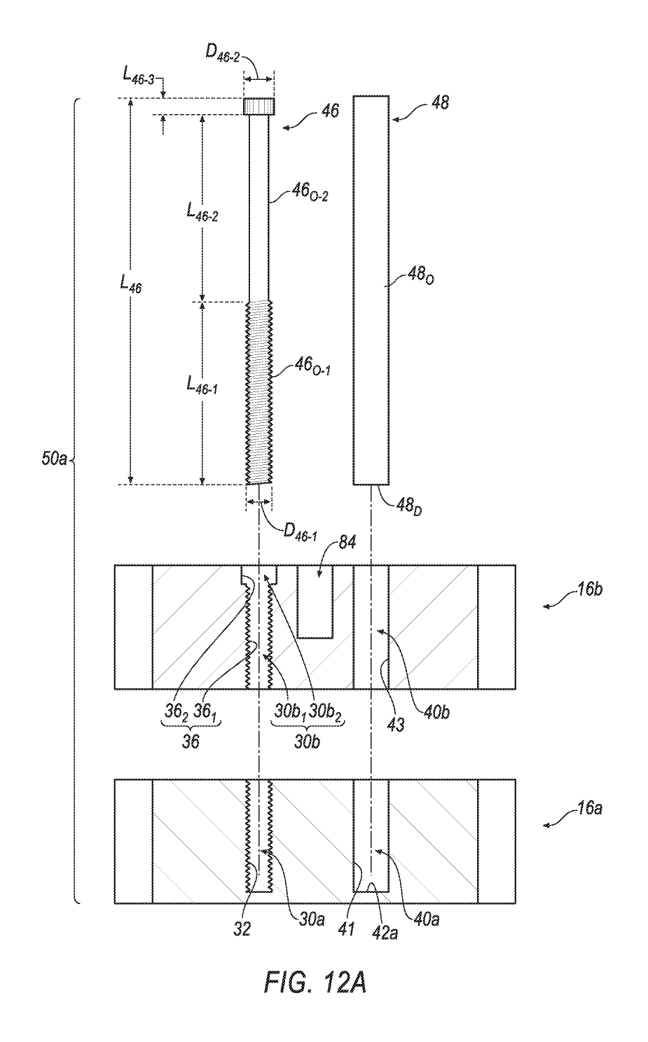

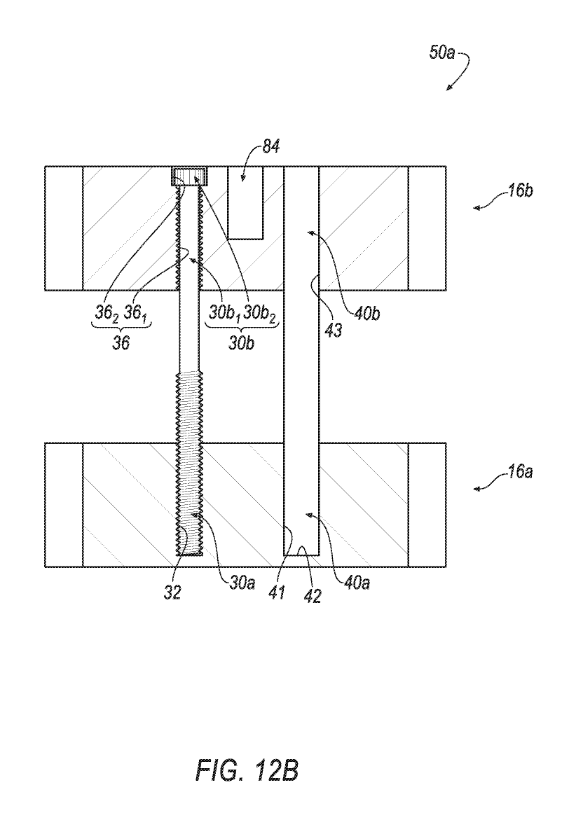

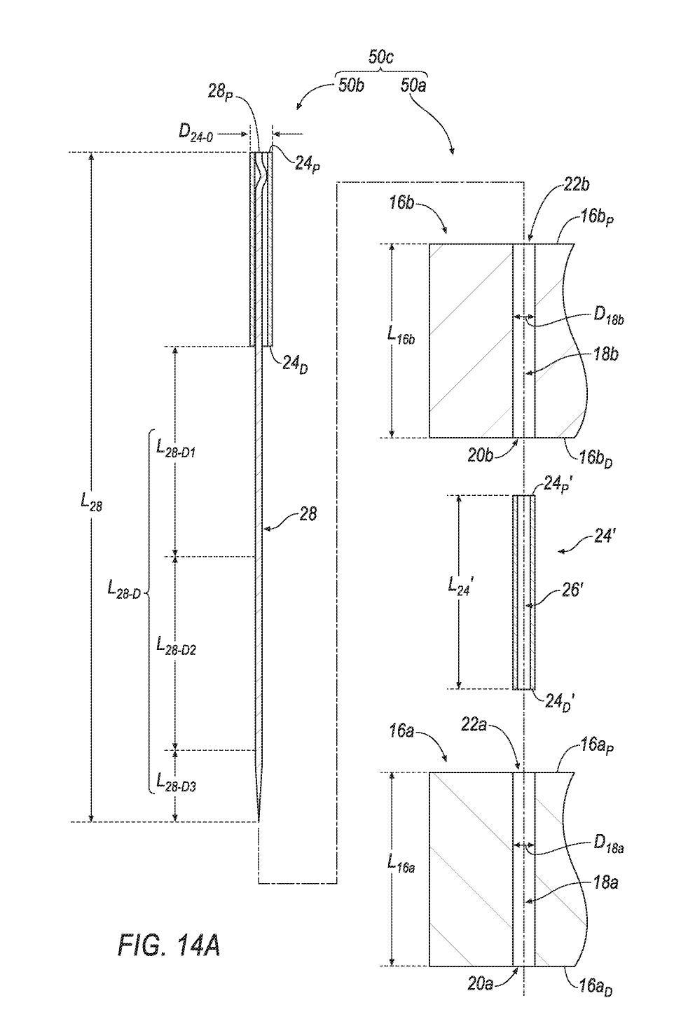

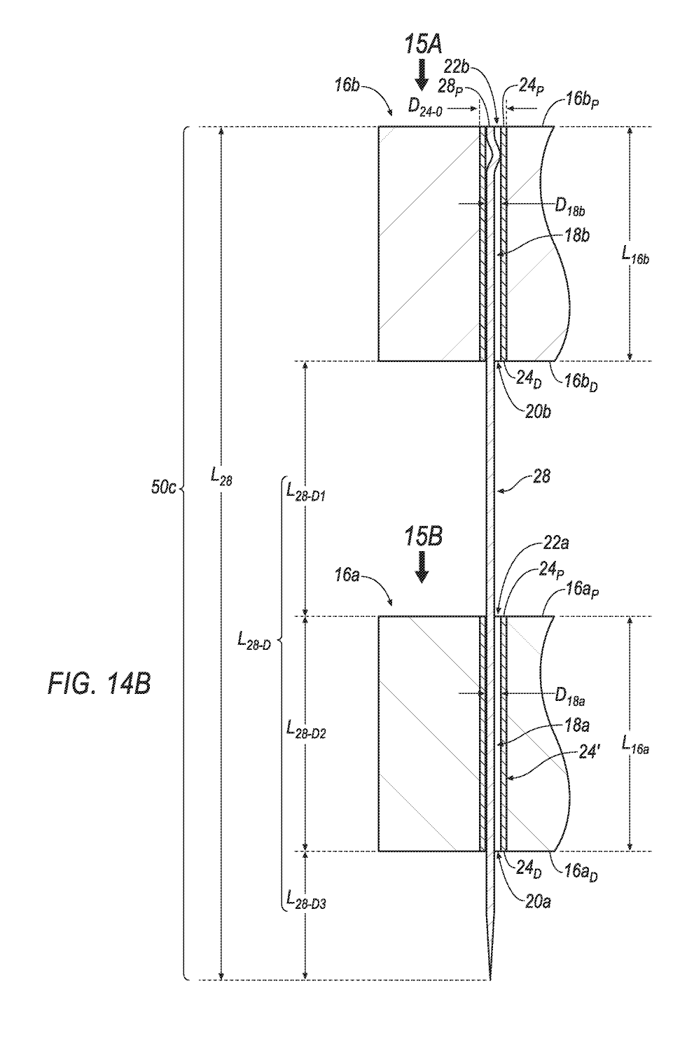

Referring to FIGS. 12A-12B, 13A-13B and 14A-14B, exemplary subassemblies of the BCI 10 including, for example two or more of: (1) the micro-electrode guide portion 16a; (2) the micro-electrode retainer portion 16b; (3) the at least one micro-electrode-containing tube 24; (4) the at least one micro-electrode 28; (5) the at least one spatial-adjuster-component 46; and (6) the at least one spatial-adjuster-guide-post 48 are shown generally at 50a (see, e.g., FIGS. 12A-12B), 50b (see, e.g., FIGS. 13A-13B) and 50c (see, e.g., FIGS. 14A-14B). As seen in FIGS. 12A-12B, a first subassembly 50a of the BCI 10 includes the micro-electrode guide portion 16a, the micro-electrode retainer portion 16b, the at least one (e.g., two) spatial-adjuster-component 46 and the at least one (e.g., two) spatial-adjuster-guide-post 48. As seen in FIGS. 13A-13B, a second subassembly 50b of the BCI 10 includes the at least one micro-electrode-containing tube 24 and the at least one micro-electrode 28. As seen in FIGS. 14A-14B, after forming the first subassembly 50a and forming one or more second subassemblies 50b, the one or more second subassemblies 50b are interfaced with the first subassembly 50a for forming the third subassembly 50c.

Referring to FIGS. 12A-12B, the at least one spatial-adjuster-component 46 is axially-aligned with (see, e.g., FIG. 12A) and then subsequently disposed within (see, e.g., FIG. 12B) the at least one spatial-adjuster-component-containing passage 30a of the micro-electrode guide portion 16a and the at least one spatial-adjuster-component-containing passage 30b of the micro-electrode retainer portion 16b for contributing to the formation of the first subassembly 50a. More specifically, the threaded outer surface 46_{O-1} defined by the first portion L₄₆₋₁ of the length L₄₆ of the spatial-adjuster-component 46 is threadingly-engaged with the threaded inner surface 32 of the at least one spatial-adjuster-component-containing passage 30a. Furthermore, the non-threaded surface 46_{O-2} defined by the second portion L₄₆₋₂ of the length L₄₆ of the spatial-adjuster-component 46 may be disposed adjacent and frictionally-engaged with the non-threaded surface 36₁ of the inner surface 36 of the spatial-adjuster-component-containing passage 30b. Yet even further, as seen in FIG. 12B, in some examples, (1) the first portion L₄₆₋₁ and the second portion L₄₆₋₂ of the length L₄₆ of the spatial-adjuster-component 46 that is defined by the first outer diameter D₄₆₋₁ of the spatial-adjuster-component 46 is arranged within the at least one spatial-adjuster-component-containing passage 30a of the micro-electrode guide portion 16a and the first portion 30b₁ of the at least one spatial-adjuster-component-containing passage 30b of the micro-electrode retainer portion 16b and (2) the third portion L₄₆₋₃ of the length L₄₆ of the spatial-adjuster-component 46 that is defined by the second outer diameter D₄₆₋₂ of the spatial-adjuster-component 46 is arranged within the second portion 30b2 of the at least one spatial-adjuster-component-containing passage 30b of the micro-electrode retainer portion 16b.

With further reference to FIGS. 12A-12B, the at least one spatial-adjuster-guide-post 48 is axially-aligned with (see, e.g., FIG. 12A) and subsequently disposed within the at least one spatial-adjuster-guide-post-containing passage 40a of the micro-electrode guide portion 16a and the at least one spatial-adjuster-guide-post passage 40b of the micro-electrode retainer portion 16b (see, e.g., FIG. 12B) for further contributing to the formation of the first subassembly 50a. In some examples, the smooth, non-threaded outer surface 48_{O} of the at least one spatial-adjuster-guide-post 48 is disposed in an adjacent relationship with respect to (1) the smooth, non-threaded surface 41 of the at least one spatial-adjuster-guide-post-containing passage 40a of the micro-electrode guide portion 16a and (2) the smooth, non-threaded surface 43 of the at least one spatial-adjuster-guide-post passage 40b of the micro-electrode retainer portion 16b. Furthermore the distal surface 48D and the at least one spatial-adjuster-guide-post 48 may be disposed adjacent or in an adjacent relationship with respect to the distal end wall surface 42a of the at least one spatial-adjuster-guide-post-containing passage 40a of the micro-electrode guide portion 16a.

In view of the above-described exemplary configuration of the first subassembly 50a, the at least one spatial-adjuster-component 46 connects the micro-electrode guide portion 16a to the micro-electrode retainer portion 16b as a result of: (1) the threaded outer surface 46_{O-1} of the spatial-adjuster-component 46 being connected to the threaded inner surface 32 of the micro-electrode guide portion 16a; and (2) the non-threaded surface 46_{O-2} of the spatial-adjuster-component 46 being disposed adjacent and frictionally-engaged with the non-threaded surface 36₁ of the spatial-adjuster-component-containing passage 30b of the micro-electrode retainer portion 16b. Further, in view of the above-described exemplary configuration of the first subassembly 50a, the at least one spatial-adjuster-guide-post 48 connects the micro-electrode guide portion 16a to the micro-electrode retainer portion 16b as a result of the smooth, non-threaded outer surface 48_{O} of the at least one spatial-adjuster-guide-post 48 being respectively disposed adjacent the smooth, non-threaded surfaces 41, 43 of the at least one spatial-adjuster-guide-post-containing passages 40a, 40b of the micro-electrode guide portion 16a and the micro-electrode retainer portion 16b.

Referring to FIGS. 13A-13B, a micro-electrode 28 is axially-aligned with (see, e.g., FIG. 13A) and then subsequently disposed within (see, e.g., FIG. 13B) a micro-electrode-receiving passage 26 of a micro-electrode-containing tube 24 for contributing to the formation of the second subassembly 50b. Access to the micro-electrode-receiving passage 26 is provided by a distal opening 25a formed by the distal surface 24_{D} of the micro-electrode-containing tube 24 and a proximal opening 25b formed by the proximal surface 24_{P} of the micro-electrode-containing tube 24.

Once the micro-electrode 28 is disposed with the micro-electrode-containing tube 24, the proximal surface 28_{P} of the micro-electrode 28 is arranged within the proximal opening 25b of the micro-electrode-containing tube 24 and aligned with the proximal surface 24_{P} of the micro-electrode-containing tube 24. Furthermore, because the length L₂₈ of the micro-electrode 28 is greater than the length L₂₄ of the micro-electrode-containing tube 24, a distal portion L_{28-D} of the length L₂₈ of the micro-electrode 28 is arranged beyond the distal surface 24_{D} of the micro-electrode-containing tube 24 such that an intermediate portion 28_{I} of the micro-electrode 28 is arranged within the distal opening 25a formed by the distal surface 24_{D} of the micro-electrode-containing tube 24.

As comparatively seen in FIGS. 13A and 13B, the outer diameter D_{28-O} of the micro-electrode 28 is less than the passage diameter D_{24-P} of the micro-electrode-containing tube 24. Therefore, because the outer diameter D_{28-O} of the micro-electrode 28 is less than the passage diameter D_{24-P} of the micro-electrode-containing tube 24, in order to frictionally-retain the micro-electrode 28 within the micro-electrode-receiving passage 26 of the micro-electrode-containing tube 24 (in a selectively axially adjustable orientation as seen in FIGS. 19A-19C), a portion of the body 28_{B} of the micro-electrode 28 extending along a proximal portion L_{28-P} of the length L₂₈ of the micro-electrode 28 that extends from the proximal surface 28_{P} of the micro-electrode 28 is shaped to include a sinusoidal shape 28_{S} that deviates from an axis A₂₈-A₂₈ extending through an axial center of both of the distal tip 28_{D} and the proximal surface 28_{P} of the micro-electrode 28. The sinusoidal shape 28_{S} of the body 28_{B} of the micro-electrode 28 results in opposite surface portions (see, e.g., 28_{O-1}, 28_{O-2}, 28_{O-3}) of outer surface 28_{O} of the micro-electrode 28 contacting and frictionally-engaging opposing surface portions (see, e.g., 24_{I-1}, 24_{I-2}, 24_{I-3}) of the inner surface 24_{I} of the micro-electrode-containing tube 24.

A plurality of the second subassemblies 50b may be formed in a substantially similar manner as described above. Referring to FIGS. 14B and 15A, each second subassembly 50b of the plurality of second subassemblies 50b may then be disposed within (e.g., in a friction-fit and axially-fixed orientation) each tube-and-micro-electrode-containing passage 18b of a plurality of tube-and-micro-electrode-containing passages 18b extending through the length L_{16b} of the micro-electrode retainer portion 16b of the first subassembly 50a for forming the third subassembly 50c.

Referring to FIGS. 14A and 14B, a second subassembly 50b is axially-aligned with (see, e.g., FIG. 14A) and then subsequently disposed within (see, e.g., FIGS. 14B and 15A) a tube-and-micro-electrode-containing passage 18b of the micro-electrode retainer portion 16b of the first subassembly 50a. Access to the tube-and-micro-electrode-containing passage 18b is provided by the distal opening 20b formed by the distal end 16b_{D} of the micro-electrode retainer portion 16b or the proximal opening 22b formed by the proximal end 16b_{P} of the micro-electrode retainer portion 16b. Because the outer diameter D_{24-O} of the micro-electrode-containing tube 24 of the second subassembly 50b is less than but approximately equal to the passage diameter D_{18b} of the tube-and-micro-electrode-containing passage 18b of the of the micro-electrode retainer portion 16b of the first subassembly 50a, the micro-electrode-containing tube 24 is friction-fit in an axially-fixed orientation with respect to the tube-and-micro-electrode-containing passage 18b of the of the micro-electrode retainer portion 16b. Furthermore, as seen in FIG. 14B, because the length L₂₄ of the micro-electrode-containing tube 24 may be about the same as the length (see, e.g., L_{16b}) of the tube-and-micro-electrode-containing passage 18b extending through the micro-electrode retainer portion 16b: (1) the distal surface 24_{D} of the micro-electrode-containing tube 24 may be aligned with the distal end 16b_{D} of the micro-electrode retainer portion 16b; and (2) the proximal surface 24_{P} of the micro-electrode-containing tube 24 may be aligned with the proximal end 16b of the micro-electrode retainer portion 16b.

With reference to FIG. 14A, the distal portion L_{28-D} of the length L₂₈ of the micro-electrode 28 that is arranged beyond the distal surface 24_{D} of the micro-electrode-containing tube 24 may be further defined by: (1) a first distal portion L_{28-D1} of the length L₂₈ of the micro-electrode 28; (2) a second distal portion L_{28-D2} of the length L₂₈ of the micro-electrode 28; and (3) a third distal portion L_{28-D3} of the length L₂₈ of the micro-electrode 28. Referring to FIG. 14B, when the micro-electrode-containing tube 24 is friction-fit in an axially-fixed orientation with respect to the tube-and-micro-electrode-containing passage 18b of the of the micro-electrode retainer portion 16b as described above, the first distal portion L_{28-D1} of the length L₂₈ of the micro-electrode 28 is arranged between the proximal end 16a_{P} of the micro-electrode guide portion 16a of the first subassembly 50a and the distal end 16b_{D} of the micro-electrode retainer portion 16b of the first subassembly 50a. Furthermore, as seen in FIG. 14B, when the micro-electrode-containing tube 24 is friction-fit in an axially-fixed orientation with respect to the tube-and-micro-electrode-containing passage 18b of the of the micro-electrode retainer portion 16b as described above, the second distal portion L_{28-D2} of the length L₂₈ of the micro-electrode 28 is arranged within the micro-electrode-containing passage 18a of the micro-electrode guide portion 16a of the first subassembly 50a. Yet even further, as seen in FIG. 14B, when the micro-electrode-containing tube 24 is friction-fit in an axially-fixed orientation with respect to the tube-and-micro-electrode-containing passage 18b of the of the micro-electrode retainer portion 16b as described above, the third distal portion L_{28-D3} of the length L₂₈ of the micro-electrode 28 extends beyond the distal end 16a_{D} of the micro-electrode guide portion 16a of the first subassembly 50a.

Referring to FIGS. 14A, 14B, and 15B, the third subassembly 50c may further comprise one or more optional micro-electrode guide tubes 24′. The one or more micro-electrode guide tubs 24′ is/are similar in size and shape to the micro-electrode-containing tube 24 described above and therefore is/are not described in greater detail here. In a substantially similar manner as described above, the micro-electrode guide tube 24′ may be axially-aligned with (see, e.g., FIG. 14A) and then subsequently disposed within (see, e.g., FIGS. 14B and 15B) a micro-electrode-containing passage 18a of the micro-electrode guide portion 16a of the first subassembly 50a in a friction-fit in and axially-fixed orientation. Once arranged within the micro-electrode-containing passage 18a of the micro-electrode guide portion 16a of the first subassembly 50a, the second distal portion L_{28-D2} of the length L₂₈ of the micro-electrode 28 is arranged within the micro-electrode-receiving passage 26′ extending through the length L₂₄′ of the micro-electrode guide tube 24′ from the distal surface 24_{D}′ of the micro-electrode guide tube 24′ to the proximal surface 24_{P}′ of the micro-electrode guide tube 24′. Furthermore, because the length L₂₄′ of the micro-electrode guide tube 24′ may be about the same as the length (see, e.g., L₁₆ₐ) of the micro-electrode-containing passage 18a extending through the micro-electrode guide portion 16a: (1) the distal surface 24_{D}′ of the micro-electrode guide tube 24′ may be aligned with the distal end 16a_{D} of the micro-electrode guide portion 16a; and (2) the proximal surface 24_{P}′ of the micro-electrode guide tube 24′ may be aligned with the proximal end 16a_{P} of the micro-electrode guide portion 16a.

Referring to FIGS. 2-3 and 16G-16J, a micro-electrode retainer interface body portion of the computing resource interface portion 14 is shown generally at 52. Referring to FIG. 16G, the micro-electrode retainer interface body portion 52 includes a distal end 52_{D} and a proximal end 52_{P}. At least a portion of the distal end 52_{D} of the micro-electrode retainer interface body portion 52 defines the distal end 14_{D} of the computing resource interface portion 14. At least one biased-pin-containing passage 54 extends through all of a length L₅₂ of the micro-electrode retainer interface body portion 52 between the distal end 52_{D} of the micro-electrode retainer interface body portion 52 and the proximal end 52_{P} of the micro-electrode retainer interface body portion 52. As will be described in the following disclosure, the at least one biased-pin-containing passage 54 contains a plurality of components (e.g., at least one distal biased pin 60 (see, e.g., FIGS. 16A-16B), at least one intermediate biasing member 62 (see, e.g., FIGS. 16C-16D) and at least one proximal electrical contact 64 (see, e.g., FIGS. 16E-16F)) for forming an interface subassembly 75 (see, e.g., FIG. 16G) of the computing resource interface portion 14.

With continued reference to FIG. 16G, at least one biased-pin-containing passage 54 extends through all of a length L₅₂ of the micro-electrode retainer interface body portion 52 between the distal end 52_{D} of the micro-electrode retainer interface body portion 52 and the proximal end 52_{P} of the micro-electrode retainer interface body portion 52. A first portion 54₁ of the at least one biased-pin-containing passage 54 is defined by a first portion 56₁ of an inner surface 56 that defines a first biased-pin-containing passage diameter D₅₄₋₁ of the at least one biased-pin-containing passage 54. A second portion 54₂ of the at least one biased-pin-containing passage 54 is defined by a second portion 56₂ of the inner surface 56 that defines a second biased-pin-containing passage diameter D₅₄₋₂ of the at least one biased-pin-containing passage 54. A third portion 54₃ of the at least one biased-pin-containing passage 54 is defined by a third portion 56₃ of the inner surface 56 that defines a third biased-pin-containing passage diameter D₅₄₋₃ of the at least one biased-pin-containing passage 54. A fourth portion 54₄ of the at least one biased-pin-containing passage 54 is defined by a fourth portion 56₄ of the inner surface 56 that defines a fourth biased-pin-containing passage diameter D₅₄₋₄ of the at least one biased-pin-containing passage 54. A fifth portion 54₅ of the at least one biased-pin-containing passage 54 is defined by a fifth portion 56₅ of the inner surface 56 that defines a fifth biased-pin-containing passage diameter D₅₄₋₅ of the at least one biased-pin-containing passage 54.

The first portion 54₁ of the at least one biased-pin-containing passage 54 defined by the first biased-pin-containing passage diameter D₅₄₋₁ of the at least one biased-pin-containing passage 54 extends along a first portion L₅₂₋₁ of the length L₅₂ from the distal end 52_{D} of the micro-electrode retainer interface body portion 52. The first biased-pin-containing passage diameter D₅₄₋₁ may be defined by a substantially constant diameter.

The second portion 54₂ of the at least one biased-pin-containing passage 54 defined by the second biased-pin-containing passage diameter D₅₄₋₂ of the at least one biased-pin-containing passage 54 extends along a second portion L₅₂₋₂ of the length L₅₂ from the first biased-pin-containing passage diameter D₅₄₋₁ and toward the proximal end 52_{P} of the micro-electrode retainer interface body portion 52. The second biased-pin-containing passage diameter D₅₄₋₂ is greater than the first biased-pin-containing passage diameter D₅₄₋₁ and progressively increases in diameter as the second biased-pin-containing passage diameter D₅₄₋₂ extends toward the proximal end 52_{P} of the micro-electrode retainer interface body portion 52.

The third portion 54₃ of the at least one biased-pin-containing passage 54 defined by the third biased-pin-containing passage diameter D₅₄₋₃ of the at least one biased-pin-containing passage 54 extends along a third portion L₅₂₋₃ of the length L₅₂ from the second biased-pin-containing passage diameter D₅₄₋₂ and toward the proximal end 52_{P} of the micro-electrode retainer interface body portion 52. The third biased-pin-containing passage diameter D₅₄₋₃ is about the same as the greatest diameter of the second biased-pin-containing passage diameter D₅₄₋₂ and may be defined by a substantially constant diameter as the third biased-pin-containing passage diameter D₅₄₋₃ extends toward the proximal end 52_{P} of the micro-electrode retainer interface body portion 52.

The fourth portion 54₄ of the at least one biased-pin-containing passage 54 defined by the fourth biased-pin-containing passage diameter D₅₄₋₄ of the at least one biased-pin-containing passage 54 extends along a fourth portion L₅₂₋₄ of the length L₅₂ from the third biased-pin-containing passage diameter D₅₄₋₃ and toward the proximal end 52_{P} of the micro-electrode retainer interface body portion 52. The fourth biased-pin-containing passage diameter D₅₄₋₄ is greater than the third biased-pin-containing passage diameter D₅₄₋₃ and progressively increases in diameter as the fourth biased-pin-containing passage diameter D₅₄₋₄ extends toward the proximal end 52 of the micro-electrode retainer interface body portion 52.

The fifth portion 54₅ of the at least one biased-pin-containing passage 54 defined by the fifth biased-pin-containing passage diameter D₅₄₋₅ of the at least one biased-pin-containing passage 54 extends along a fifth portion L₅₂₋₅ of the length L₅₂ from the fourth biased-pin-containing passage diameter D₅₄₋₄ and toward the proximal end 52_{P} of the micro-electrode retainer interface body portion 52. The fifth biased-pin-containing passage diameter D₅₄₋₅ is about the same as the greatest diameter of the fourth biased-pin-containing passage diameter D₅₄₋₄ and may be defined by a substantially constant diameter as the fifth biased-pin-containing passage diameter D₅₄₋₅ extends toward the proximal end 52_{P} of the micro-electrode retainer interface body portion 52.

Access to the at least one biased-pin-containing passage 54 is permitted by a distal opening 58a formed by the distal end 52_{D} of the micro-electrode retainer interface body portion 52 and a proximal opening 58b formed by the proximal end 52_{P} of the micro-electrode retainer interface body portion 52. The micro-electrode retainer interface body portion 52 defines a plurality of biased-pin-containing passages 54. Furthermore, the plurality of biased-pin-containing passages 54 may be sub-divided or grouped into a plurality of arrays of plurality of biased-pin-containing passages 54. With reference to FIGS. 16G-16H, the interface subassembly 75 is arranged within the biased-pin-containing passage 54 of the micro-electrode retainer interface body portion 52.

Referring to FIGS. 16A and 16B, the at least one distal biased pin 60 includes a body 60a having: (1) a distal micro-electrode-containing-tube-contacting portion 60a₁; (2) an intermediate shoulder portion 60a2 connected to the distal micro-electrode-containing-tube-contacting portion 60a₁; and (3) a proximal biasing-member-contacting portion 60a3 connected to the intermediate shoulder portion 60a2. Furthermore, the body 60a is defined by a length L₆₀ₐ (see, e.g., FIG. 16B) extending between a distal end 60a_{D} of the body 60a and a proximal end 60a_{P} of the body 60a.

As seen in FIG. 16B, the body 60a is further defined by an outer surface 61 defining a non-constant diameter along the length L₆₀ₐ of the body 60a. The outer surface 61 is defined by a first outer surface portion 61₁ extending away from the distal end 60a_{D} of the body 60a, a second outer surface portion 61₂ extending away from the first outer surface portion 61₁ and a third outer surface portion 61₃ extending away from the second outer surface portion 61₂. The non-constant diameter is defined by: (1) a first diameter D₆₀ₐ₋₁ extending along a first portion L₆₀ₐ₋₁ of the length L₆₀ₐ of the body 60a defined by the first outer surface portion 61₁ of the outer surface 61; (2) a second diameter D₆₀ₐ₋₂ extending along a second portion L₆₀ₐ₋₂ of the length L₆₀ₐ of the body 60a defined by the second outer surface portion 61₂ of the outer surface 61; and (3) a third diameter D₆₀ₐ₋₃ extending along a third portion L₆₀ₐ₋₃ of the length L₆₀ₐ of the body 60a defined by the third outer surface portion 61₃ of the outer surface 61.

The first diameter D₆₀ₐ₋₁ extending along the first portion L₆₀ₐ₋₁ of the length L₆₀ₐ of the body 60a may be defined by a substantially constant diameter. The second diameter D₆₀ₐ₋₂ extending along the second portion L₆₀ₐ₋₂ of the length L₆₀ₐ of the body 60a is greater than the first diameter D₆₀ₐ₋₁ extending along the first portion L₆₀ₐ₋₁ of the length L₆₀ₐ of the body 60a and progressively increases in diameter as the second portion L₆₀ₐ₋₂ of the length L₆₀ₐ of the body 60a extends toward the proximal end 60a_{P} of the body 60. The third diameter D₆₀ₐ₋₃ extending along the third portion L₆₀ₐ₋₃ of the length L₆₀ₐ of the body 60a is about the same as the greatest diameter of the second diameter D₆₀ₐ₋₂ extending along the second portion L₆₀ₐ₋₂ of the length L₆₀ₐ of the body 60a and may be defined by a substantially constant diameter as the third portion L₆₀ₐ₋₃ of the length L₆₀ₐ of the body 60a extends toward the proximal end 60a_{P} of the body 60.

Referring to FIGS. 16C and 16D, the least one intermediate biasing member 62 may be defined by a coil spring. The coil spring 62 may be defined by a body 66 having a length L₆₂ extending between a distal end 66_{D} of the body 66 and a proximal end 66 of the body 66. Furthermore, the body 66 may be defined by an outer surface 66_{O} and an inner surface 66_{I}. The inner surface 66_{I} defines a passage 68 extending through the body 66. The outer surface 66_{O} defines an outer diameter D_{66-O} of the body 66. The inner surface 66_{I} defines a passage diameter D₆₈ of the passage 68 extending through the body 66.

Referring to FIGS. 16E-16F, the at least one proximal electrical contact 64 is defined by a body 70 having a length L₇₀ extending between a distal end 70_{D} of the body 70 and a proximal end 70_{P} of the body 70. The body 70 includes an outer surface 71 that defines a substantially constant diameter D₇₀ extending along the length L₇₀ of the body 70.

Referring to FIG. 16G, the interface subassembly 75 of the computing resource interface portion 14 is assembled as follows. Firstly, a distal biased pin 60 is arranged within a biased-pin-containing passage 54 such that the second outer surface portion 61₂ of the outer surface 61 of the body 60a of the distal biased pin 60 defined by the intermediate shoulder portion 60a2 is disposed adjacent the second portion 56₂ of the inner surface 56 that defines the second biased-pin-containing passage diameter D₅₄₋₂ of the biased-pin-containing passage 54.

Then, the coil spring 62 is arranged within the biased-pin-containing passage 54 of the micro-electrode retainer interface body portion 52 such that the distal end 66_{D} of the body 66 of the coil spring 62 is disposed adjacent the proximal end 60a_{P} of the body 60a of the distal biased pin 60. Once arranged within the biased-pin-containing passage 54, the coil spring 62 may occupy most of the third portion 54₃ of the at least one biased-pin-containing passage 54 that extends along the third portion L₅₂₋₃ of the length L₅₂ of the micro-electrode retainer interface body portion 52.

Then, the proximal electrical contact 64 is arranged within the biased-pin-containing passage 54 of the micro-electrode retainer interface body portion 52 such that the distal end 70_{D} of the body 70 of the proximal electrical contact 64 is disposed adjacent the proximal end 66_{P} of the body 66 of the coil spring 62. Furthermore, once arranged in the biased-pin-containing passage 54 of the micro-electrode retainer interface body portion 52: (1) a portion of the distal end 70_{D} of the body 70 is disposed adjacent the fourth portion 56₄ of the inner surface 56 that defines the fourth biased-pin-containing passage diameter D₅₄₋₄ of the biased-pin-containing passage 54; and (2) a portion of the outer surface 71 of the body 70 is disposed adjacent the fifth portion 56₅ of the inner surface 56 that defines the fifth biased-pin-containing passage diameter D₅₄₋₅ of the biased-pin-containing passage 54.

Referring to FIG. 16H, as a result of the arrangement of the proximal electrical contact 64 being arranged within the biased-pin-containing passage 54 of the micro-electrode retainer interface body portion 52 as described above: (A) the body 70 of the proximal electrical contact 64 is arranged in a friction-fit and axially-fixed relationship within the biased-pin-containing passage 54 of the micro-electrode retainer interface body portion 52; and (B) the coil spring 62 is axially compressed between the distal biased pin 60 and the proximal electrical contact 64. As a result of the compression of the coil spring 62 as described above: (1) the second outer surface portion 61₂ of the outer surface 61 of the body 60a of the distal biased pin 60 is seated adjacent the second portion 56₂ of the inner surface 56 that defines the second biased-pin-containing passage diameter D₅₄₋₂ of the biased-pin-containing passage 54; and (2) the distal biased pin 60 is axially urged by the coil spring 62 through the distal opening 58a formed by the distal end 52_{D} of the micro-electrode retainer interface body portion 52 such that a portion L₆₀ₐ₋₁ₐ (see, e.g., FIGS. 16B and 16H) of the first portion L₆₀ₐ₋₁ of the length L₆₀ₐ of the body 60a of the distal biased pin 60 extends beyond the distal end 52_{D} of micro-electrode retainer interface body portion 52. Furthermore, the length L₇₀ of the body 70 of at least one proximal electrical contact 64 may be selectively sized relative to: (1) the fifth portion 54₅ of the at least one biased-pin-containing passage 54 extending along the fifth portion L₅₂₋₅ of the length L₅₂ of the micro-electrode retainer interface body portion 52; and (2) at least a portion of the fourth portion 54₄ of the at least one biased-pin-containing passage 54 extending along the fourth portion L₅₂₋₄ of the length L₅₂ of the micro-electrode retainer interface body portion 52 such that a portion L₇₀₋₁ (see, e.g., FIGS. 16E and 16H) of the length L₇₀ of the body 70 of the at least one proximal electrical contact 64 extends beyond the proximal end 52_{P} of micro-electrode retainer interface body portion 52.

Although an interface subassembly 75 of the computing resource interface portion 14 has been described above at FIGS. 16G and 16H, the interface subassembly 75 is not limited to the above-described design. In an example, an alternative interface subassembly is shown generally at 75′ in FIGS. 16G′ and 16H′. Most of the surfaces, geometries and the like of the components of the interface subassembly 75′ are similar to the components of the interface subassembly 75 and therefore will not be described in detail here (i.e., the surfaces, geometries and the like are identified with a prime symbol (′) next to corresponding reference numerals). Furthermore, other structural components, surfaces and the like associated with the interface subassembly 75′ at FIGS. 16A′-16J′ are substantially similar to the structural components, surfaces and the like of FIGS. 16A-16J, and therefore, are also not described in greater detail here (i.e., the structural components, surfaces and the like are identified with a prime symbol (′) next to corresponding reference numerals).

In an example, the interface subassembly 75′ differs from the interface subassembly 75 in that: (1) a passage 72′ (see, e.g., FIGS. 16A′ and 16B′) extends through all of the length L₆₀ₐ′ between the distal end 60a_{D}′ of the body 60a′ and the proximal end 60a_{P}′ of the body 60a′ of the distal biased pin 60′; and (2) a passage 76′ (see, e.g., FIGS. 16E′ and 16F′) extends through all of the length L₇₀′ between the distal end 70_{D}′ of the body 70′ and the proximal end 70_{P}′ of the body 70a′ of the proximal electrical contact 64′. Access to the passage 72′ is permitted by a distal opening 74a′ (see, e.g., FIGS. 16A′ and 16B′) formed by the distal end 60a_{D}′ of the distal biased pin 60′ or a proximal opening 74b′ (see, e.g., FIG. 16B′) formed by the proximal end 60a′ of the distal biased pin 60′. Access to the passage 76′ is permitted by a distal opening 78a′ (see, e.g., FIGS. 16E′ and 16F′) formed by the distal end 70_{D}′ of the proximal electrical contact 64′ or a proximal opening 78b′ (see, e.g., FIG. 16F′) formed by the proximal end 70_{P}′ of the proximal electrical contact 64′.

As seen in FIGS. 16H′ and 16I′, the passages 72′, 76′ of the distal biased pin 60′ and the proximal electrical contact 64′ in combination with the passage 68′ extending through the intermediate biasing member 62′ collectively forms at least one micro-electrode access passage 80′ extending through the interface subassembly 75′. Furthermore, the at least one micro-electrode access passage 80′ extending through the interface subassembly 75′ is axially aligned with the micro-electrode-receiving passage 26 of the micro-electrode-containing tube 24 in order to permit access to the proximal surface 28_{P} of the at least one micro-electrode 28 contained within the micro-electrode-receiving passage 26 of the micro-electrode-containing tube 24. The purpose of the at least one micro-electrode access passage 80′ extending through the interface subassembly 75′ will be described in greater detail in the following disclosure in association with FIGS. 19A-19C.

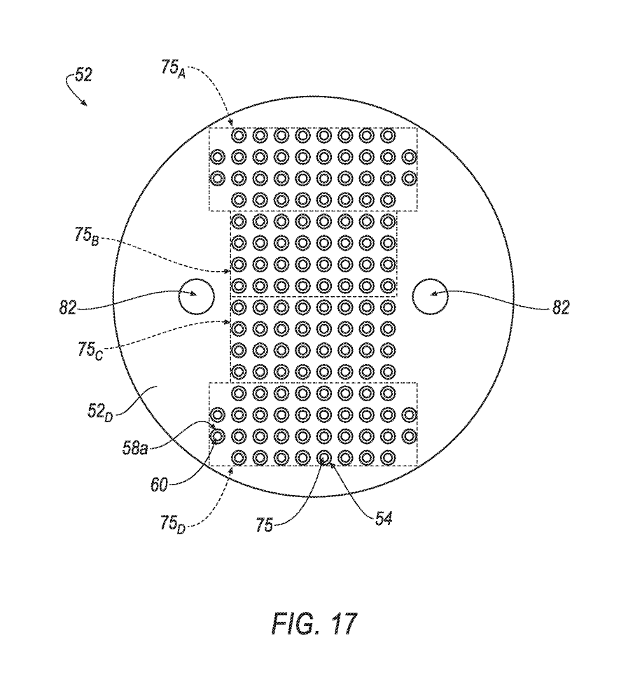

Referring to FIGS. 16H and 17, the distal end 52_{D} of the micro-electrode retainer interface body portion 52 is shown. Each interface subassembly 75 (of the plurality of interface subassemblies 75) including a distal biased pin 60, an intermediate biasing member 62 and a proximal electrical contact 64 is disposed within a biased-pin-containing passage 54 (of the plurality of biased-pin-containing passages 54) of the micro-electrode retainer interface body portion 52. In an example, as seen in FIG. 17, the plurality of interface subassemblies 75 may be sub-divided or grouped into a plurality of arrays of a plurality of interface subassemblies 75_{A}, 75_{B}, 75_{C}, 75_{D}.

With reference to FIGS. 15A and 16H, the proximal end 16b_{P} of the micro-electrode retainer portion 16b is shown. Each second subassembly 50b (of the plurality of second subassemblies 50b) of the BCI 10 including the at least one micro-electrode 28 disposed within the at least one micro-electrode-containing tube 24 is disposed within a tube-and-micro-electrode-containing passage 18b (of the plurality of tube-and-micro-electrode-containing passages 18b) of the micro-electrode retainer portion 16b. In an example, the plurality of second subassemblies 50b may be sub-divided or grouped into a plurality of arrays of a plurality of second subassemblies 50b_{A}, 50b_{B}, 50b_{C}, 50b_{D} (see, e.g., FIG. 15A).

Referring to FIGS. 3 and 17, at least one (e.g. a pair of) male connector portions of the micro-electrode retainer interface body portion of the computing resource interface portion 14 is shown generally at 82. The at least one male connector portion 82 of the computing resource interface portion 14 extends away from the distal end 52_{D} of the body portion 52 of the computing resource interface portion 14. Furthermore, as seen in FIGS. 3, 5B, 12A-12B and 15A, at least one (e.g., a pair of) female connector portions of the spatially-adjustable animalia-engaging portion 12 is shown generally at 84. The at least one female connector portion 84 of the spatially-adjustable animalia-engaging portion 12 is formed by the micro-electrode retainer portion 16b and extends into the micro-electrode retainer portion 16b from the proximal end 16b_{P} of the micro-electrode retainer portion 16b at a distance approximately equal to a length of the at least one male connector portion 82 of the computing resource interface portion 14.

As seen in FIG. 3, the at least one male connector portion 82 of the computing resource interface portion 14 corresponds to and is/are axially-aligned with the at least one female connector portion 84 of the spatially-adjustable animalia-engaging portion 12. Subsequently, the at least one male connector portion 82 is/are disposed within the at least one female connector portion 84 for mechanically-connecting the distal end 14_{D} of the computing resource interface portion 14 to the proximal end 12_{P} of the spatially-adjustable animalia-engaging portion 12.

As seen in FIGS. 3 and 16H, upon axially-aligning the at least one male connector portion 82 with the at least one female connector portion 84, each interface subassembly 75 (including a distal biased pin 60) of the plurality of interface subassemblies 75 is/are axially aligned with a corresponding second subassembly 50b (including the at least one micro-electrode-containing tube 24 and the at least one micro-electrode 28) of the plurality of second subassemblies 50b. Referring to FIG. 16I, when the computing resource interface portion 14 is connected to the spatially-adjustable animalia-engaging portion 12 by arranging the at least one male connector portion 82 within the at least one female connector portion 84, the distal end 60a_{D} of the body 60a of the distal biased pin 60 of each interface subassembly 75 of the plurality of interface subassemblies 75 is disposed adjacent the proximal surface 24_{P} of the micro-electrode-containing tube 24 and the proximal surface 28_{P} of the at least one micro-electrode 28 of each second subassembly 50b of the plurality of second subassemblies 50b for electrically-connecting the distal end 14_{D} of the computing resource interface portion 14 to the proximal end 12_{P} of the spatially-adjustable animalia-engaging portion 12.

With reference to FIG. 16H and as described above, when the distal end 14_{D} of the computing resource interface portion 14 is not connected to the proximal end 12_{P} of the spatially-adjustable animalia-engaging portion 12, the coil spring 62 is arranged in an expanded state such that the distal biased pin 60 is axially urged by the coil spring 62 through the distal opening 58a formed by the distal end 52_{D} of the micro-electrode retainer interface body portion 52 such that the portion L₆₀ₐ₋₁ₐ (see, e.g., FIGS. 16B and 16H) of the first portion L₆₀ₐ₋₁ of the length L₆₀ₐ of the body 60a of the distal biased pin 60 extends beyond the distal end 52_{D} of micro-electrode retainer interface body portion 52. With reference to FIG. 16I, thereafter, when the distal end 14_{D} of the computing resource interface portion 14 is connected to the proximal end 12_{P} of the spatially-adjustable animalia-engaging portion 12, the bias provided by the coil spring 62 is overcome for arranging the first portion L₆₀ₐ₋₁ of the length L₆₀ₐ of the body 60a of the distal biased pin 60 within the micro-electrode retainer interface body portion 52 such that the first portion L₆₀ₐ₋₁ of the length L₆₀ₐ of the body 60a of the distal biased pin 60 does not extend beyond the distal end 52_{D} of micro-electrode retainer interface body portion 52.

With the coil spring 62 arranged in the compressed state as seen in FIG. 16I, the distal end 60a_{D} of the body 60a of the distal biased pin 60 of each interface subassembly 75 of the plurality of interface subassemblies 75 is always urged and disposed adjacent at least the proximal surface 24_{P} of the micro-electrode-containing tube 24 of each second subassembly 50b of the plurality of second subassemblies 50b in order to always ensure electrical contact of the distal end 14_{D} of the computing resource interface portion 14 to the proximal surface 24_{P} of the micro-electrode-containing tube 24 (i.e., at least a portion of the proximal end 12_{P} of the spatially-adjustable animalia-engaging portion 12). As a result, each interface subassembly 75 of the plurality of interface subassemblies 75 continuously imparts an axial bias to the distal biased pin 60 of each interface subassembly 75 of the plurality of interface subassemblies 75 such that the distal end 60a_{D} of the body 60a of the distal biased pin 60 of each interface subassembly 75 of the plurality of interface subassemblies 75 is always urged and disposed adjacent the proximal surface 24_{P} of the micro-electrode-containing tube 24 of each second subassembly 50b of the plurality of second subassemblies 50b. Therefore, the plurality of arrays of a plurality of interface subassemblies 75_{A}, 75_{B}, 75_{C}, 75_{D} (see, e.g., FIG. 17) are, respectively, always in electrical communication with the plurality of second subassemblies 50b_{A}, 50b_{B}, 50b_{C}, 50b_{D} (see, e.g., FIG. 15A).

Referring to FIGS. 2-3 and 16J, at least one or a plurality of (e.g., approximately one-hundred-and-twenty-eight) electrical conduits of the computing resource interface portion 14 is shown generally at 86. Each electrical conduit 86 includes a distal end 86_{D} (see, e.g., FIGS. 2 and 16J) and a proximal end 86_{P} (see, e.g., FIG. 2).

Referring to FIG. 16J, the at least one electrical conduit 86 includes a conductive portion 86a (e.g., a wire) and an insulator portion 86b (e.g., an outer jacket) circumscribing the conductive portion 86a along the entire length of the conductive portion 86a. Furthermore, as seen in FIG. 16J, at least a distal surface 86a_{D} (that at least partially defines the distal end 86_{D} of electrical conduit 86) of each conductive portion 86a of the plurality of electrical conduits 86 is disposed adjacent the distal end 70_{D} of the body 70 of the proximal electrical contact 64 of each interface subassembly 75 of the plurality of interface subassemblies 75.

With reference to FIG. 2, the computing resource interface portion 14 may be further defined by a circuit board plug 90a/90b/90c/90d including one or a plurality of (e.g., collectively, approximately one-hundred-and-twenty-eight) of terminals 88. The circuit board plug 90a, 90b, 90c, 90d may be commercially available from OMNETICS®. A proximal surface (not shown) that at least partially defines the proximal end of each electrical conduit 86 is connected to each terminal 88 of the plurality of terminals 88 of the circuit board plug 90a/90b/90c/90d. Furthermore, each terminal 88 of the plurality of terminals 88 of the circuit board plug 90a/90b/90c/90d may generally define the proximal end 14_{P} of the computing resource interface portion 14. Upon connecting the circuit board plug 90a/90b/90c/90d to a circuit board of the computing resource C (which may include, for example one or more neuronal amplifiers, such as, for example, a miniature INTAN TECHNOLOGIES® integrated one-hundred-and-twenty-eight channel pre-amplifier), the computing resource C may be placed in electrical communication with the spatially-adjustable animalia-engaging portion 12 by way of the computing resource interface portion 14.

In an example, each circuit board plug 90a/90b/90c/90d includes thirty-two terminals 88. Furthermore, as seen in FIG. 2, the plurality of electrical conduits 86 may be sub-divided or grouped into a plurality of arrays of a plurality of electrical conduits 86_{A}, 86_{B}, 86_{C}, 86_{D}. As seen in FIG. 2, each array of electrical conduits 86_{A}, 86_{B}, 86_{C}, 86_{D} is exclusively associated with each circuit board plug 90a/90b/90c/90d. Yet even further, each array of electrical conduits 86_{A}, 86_{B}, 86_{C}, 86_{D} may include thirty-two electrical conduits 86 whereby each electrical conduit 86 of the thirty-two electrical conduits 86 corresponds to each terminal 88 of the thirty-two terminals 88.

In an example, each array of interface subassemblies 75_{A}, 75_{B}, 75_{C}, 75_{D} includes thirty-two interface subassemblies 75 (thereby collectively defining a total of one-hundred-and-twenty-eight interface subassemblies 75 of the spatially-adjustable animalia-engaging portion 12). Furthermore, each array of interface subassemblies 75_{A}, 75_{B}, 75_{C}, 75_{D} may be exclusively associated with each array of electrical conduits 86_{A}, 86_{B}, 86_{C}, 86_{D} including the thirty-two electrical conduits described above. Furthermore, in an example, each array of second subassemblies 50b_{A}, 50b_{B}, 50b_{C}, 50b_{D} includes thirty-two second subassemblies 50b (thereby collectively defining a total of one-hundred-and-twenty-eight second subassemblies 50b of the spatially-adjustable animalia-engaging portion 12). Furthermore, each array of second subassemblies 50b_{A}, 50b_{B}, 50b_{C}, 50b_{D} may be exclusively associated with each array of interface subassemblies 75_{A}, 75_{B}, 75_{C}, 75_{D} including the thirty-two subassemblies described above.

In view of the above-described exemplary example of arrays, the BCI 10 may define one-hundred-and-twenty-eight electrical communication channels (and four ground channels) that are grouped or subdivided into a first array of electrical communication channels (see, e.g., reference numerals 50b_{A}, 75_{A}, 86_{A}, 90a), a second array of electrical communication channels (see, e.g., reference numerals 50b_{B}, 75_{B}, 86_{B}, 90b), a third array of electrical communication channels (see, e.g., reference numerals 50b_{C}, 75_{C}, 86_{C}, 90c), and a fourth array of electrical communication channels (see, e.g., reference numerals 50b_{D}, 75_{D}, 86_{D}, 90d). In an example, each communication channel of the arrays of communication channels (see, e.g., 50b_{A}, 50b_{B}, 50b_{C}, 50b_{D}, 75_{A}, 75_{B}, 75_{C}, 75_{D}) formed by the spatially-adjustable animalia-engaging portion 12 may be spaced apart from one another by approximately about 0.4 mm. Although an exemplary implementation of the BCI 10 is directed to one-hundred-and-twenty-eight electrical communication channels that are grouped into four arrays, the BCI 10 is not limited to such a configuration. For example, the BCI 10 may include as few as one channel or as many as one-thousand or more channels that are grouped or sub-divided into any desirable number of arrays.

Referring to FIGS. 18A and 18B, an exemplary implementation for operating the BCI 10 is described. Firstly, as seen in FIG. 18A, the plurality of micro-electrodes 28 of the plurality of second sub-assemblies 50b of the spatially-adjustable animalia-engaging portion 12 are shown arranged in a first orientation whereby a portion of the length L₂₈ of the plurality of micro-electrodes 28 are shown in a default orientation, whereby each micro-electrode 28 of the plurality of micro-electrodes 28 are extended from the distal end 16a_{D} of the micro-electrode guide portion 16a at a similar distance D_{28-B1}, which may be approximately equal to distance ranging between 1 mm and 2 mm. The distance D_{28-B1} may be sufficient enough for at least the distal tip 28_{D} of each micro-electrode 28 to be arranged within the brain B of the animalia A.

As seen in FIG. 18A, an actuator 92 (e.g., a motor) may be disposed within the micro-electrode retainer interface body portion 52. The actuator 92 may be communicatively-coupled to the computing resource C and mechanically-interfaced (e.g., frictionally-coupled or keyed) with the third portion 46_{O-3} of the outer surface 46_{O} of the spatial-adjuster-component 46. With reference to FIG. 18B, upon receiving a signal from the computing resource C, the actuator 92 may impart rotational motion R to the spatial-adjuster-component 46. As a result of the threaded connection of the first portion 46_{O-1} of the outer surface 46_{O} of the spatial-adjuster-component 46 with the inner surface 32 of the micro-electrode guide portion 16a, the rotational motion R of the spatial-adjuster-component 46 results in the portion of the length L₂₈ of the plurality of micro-electrodes 28 being further extended from the distal end 16a_{D} of the micro-electrode guide portion 16a at a similar distance D_{28-B2} for further extending the plurality of micro-electrodes 28 into the brain B of the animalia A.

While still attached to the brain B of the animalia A, an operator may desire further spatial adjustment of one micro-electrode 28 of the plurality of micro-electrodes 28 (due to brain neurons not being evenly distributed in the brain B and therefore not providing neuronal activity to one or more micro-electrodes 28). Firstly, as seen in FIGS. 18C and 19A, in an example, the operator may disconnect the micro-electrode retainer interface body portion 52 from the micro-electrode retainer portion 16b (e.g., by removing the at least one male connector portion 82 from the at least one female connector portion 84) in order to obtain access to the proximal surface 28_{P} of each micro-electrode 28 of the plurality of micro-electrodes 28. Thereafter, as seen in FIG. 19B, the operator may arrange a distal end surface PD of a push-pin P adjacent a proximal surface 28_{P} of a micro-electrode 28 for imparting an axial force to the proximal surface 28_{P} of a micro-electrode 28 such that the push-pin P may be inserted through the proximal opening 25b formed by the proximal surface 25_{P} of the micro-electrode-containing tube 24 and into the micro-electrode-receiving passage 26 for axially adjusting the orientation of the micro-electrode 28 relative to the micro-electrode-containing tube 24 for further extending the micro-electrode 28 from the distal end 16a_{D} of the micro-electrode guide portion 16a at a distance D_{28-B3} (see, e.g., FIG. 18C) that is greater than the default distance D_{28-B1} or the further-extended distance D_{28-B2} for further extending the micro-electrode 28 into the brain B of the animalia A. In an example, the distance D_{28-B3}, which may be a maximum distance that the micro-electrode is permitted to be extended from the distal end 16a_{D} of the micro-electrode guide portion 16a, may be approximately equal to about 9 mm.

As seen in FIG. 19C, with the push-pin P removed from the micro-electrode-receiving passage 26 for axially adjusting the orientation of the micro-electrode 28, the operator may re-connect the micro-electrode retainer interface body portion 52 to the micro-electrode retainer portion 16b (e.g., by re-disposing the at least one male connector portion 82 in the at least one female connector portion 84). With reference to FIG. 19C, even after the micro-electrode 28 is axially displaced with respect to the micro-electrode-containing tube 24, the distal biased pin 60 is still in electrical communication with the second subassembly 50b as a result of the distal end 60a_{D} of the body 60a of the distal biased pin 60 being disposed adjacent the proximal surface 24_{P} of the micro-electrode-containing tube 24.

Although a micro-electrode 28 may be axially displaced relative a micro-electrode-containing tube 24 as described above at FIGS. 19A-19C by disconnecting the micro-electrode retainer interface body portion 52 from the micro-electrode retainer portion 16b, the above-described methodology is an exemplary implementation. In other examples, a micro-electrode 28 may be axially displaced relative a micro-electrode-containing tube 24 without disconnecting the micro-electrode retainer interface body portion 52 from the micro-electrode retainer portion 16b. With reference to FIG. 16I′, when the distal end 86_{D} of electrical conduit 86 is disconnected from the proximal electrical contact 64′, a push-pin P may be inserted through the at least one micro-electrode access passage 80′ extending through the interface subassembly 75′ for axially-engaging the proximal surface 28_{P} of a micro-electrode 28 in a substantially similar manner as described above at FIG. 19B. Furthermore, although a manual axial adjustment of a micro-electrode 28 relative a micro-electrode-containing tube 24 may be conducted with, for example, a push-pin P, other implementations of the BCI 10 may include, for example, the actuator 92 including structure (not shown) for axially engaging any one of the micro-electrodes 28 for spatially adjusting any of the micro-electrodes 28 without use of, for example, a push-pin P or requiring the disassembly or disconnection of any portion of the BCI 10 as described above.

After the BCI 10 is interfaced with (and, in some instances, selectively spatially manipulated by an operator) the animalia's brain B as described above, the BCI 10 may be utilized for measuring brain signals B_{S} of brain neurons of the animalia's brain B. The brain signals B_{S} may be sent from the brain B and to the computing resource C for viewing by the operator and subsequent electronic storage in memory of the computing resource C. Furthermore, the computing resource C may send signals to the animal's brain B by way of the BCI 10 in order to, for example, stimulate the animal's brain B in order to, for example, cause movement of any body part (e.g., a limb) of the animal A, or, alternatively, treat neurological disorders. In other examples, the BCI 10 may be further modified, or, alternatively, cooperate with one or more other devices (e.g., the computing resource C and a fluid delivery system) in order to perform, for example, microfluidic delivery and optogenetic delivery to the brain B during the study of the neuronal activity of the brain B.

Furthermore, although a computing resource interface portion 14 in the above-described embodiments includes a wired connection for sending brain signals B_{S} to the computing resource C, other implementations may include a wireless connection. For example, rather than utilizing one or more electrical conduits 86, the one or more electrical conduits may be replaced with a headstage (not shown, which may be commercially available from TBSI®) including, for example, one-hundred and-twenty-eight channels. Such configurations may also include micro-bonding one or more sixty-four channel micro-pre-amplifiers (not shown, which may be commercially available from INTAN TECHNOLOGIES®) that are stacked and packaged together in a tethered connection in order to provide more than one array including more than one-thousand channels; because the pre-amplifier includes an embedded multiplex electronic system, the output of the pre-amplifier includes ten-to-twenty pins for providing one-hundred-and-twenty-eight channels that provides the potential to expand the number of channels to: two-hundred-and-fifty-six channels, five-hundred-and-twelve channels or one-thousand-and-twenty-four channels.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Implementations of the subject matter and the functional operations described in this specification can be implemented in digital electronic circuitry, or in computer software, firmware, or hardware, including the structures disclosed in this specification and their structural equivalents, or in combinations of one or more of them. Moreover, subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The terms “data processing apparatus”, “computing device” and “computing processor” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as an application, program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

One or more aspects of the disclosure can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

What is claimed is:

1. A subassembly of a spatially-adjustable animalia-engaging portion of a brain-computer interface, the subassembly comprising:
a micro-electrode-containing tube defined by a distal surface, a proximal surface, an outer surface and an inner surface, wherein the micro-electrode-containing tube is defined by a length extending between the distal surface and the proximal surface, wherein the inner surface of the micro-electrode-containing tube defines a micro-electrode-receiving passage having a passage diameter extending through the length of the micro-electrode-containing tube from the distal surface to the proximal surface, wherein access to the micro-electrode-receiving passage is provided by a distal opening formed by the distal surface of the micro-electrode-containing tube and a proximal opening formed by the proximal surface of the micro-electrode-containing tube, wherein the outer surface of the micro-electrode-containing tube defines an outer diameter of the micro-electrode-containing tube; and
a micro-electrode disposed within the micro-electrode-receiving passage of the micro-electrode-containing tube, wherein the at least one micro-electrode is defined by a distal tip a proximal surface and an outer surface, wherein the at least one micro-electrode is further defined by a length extending between the distal tip and the proximal surface of the micro-electrode, wherein the outer surface of the micro-electrode defines an outer diameter of the micro-electrode that is less than the passage diameter of the micro-electrode-containing tube, wherein the proximal surface of the micro-electrode is substantially aligned with the proximal surface of the micro-electrode-containing tube, wherein the distal tip of the micro-electrode is arranged beyond the distal surface of the micro-electrode-containing tube, wherein a portion of the body of the micro-electrode defined by the outer surface of the micro-electrode deviates from an axial extending through an axial center of both of the distal tip and the proximal surface of the micro-electrode whereby one or more portions of the portion of the body of the micro-electrode defined by the outer surface of the micro-electrode is disposed adjacent one or more portions of the inner surface defining the micro-electrode-receiving passage of the micro-electrode-containing tube for frictionally-fitting the micro-electrode within the micro-electrode-containing tube in an axially-adjustable orientation.

2. The subassembly of claim 1, wherein the portion of the body of the micro-electrode extends along a proximal portion of the length of the micro-electrode from the proximal surface of the micro-electrode.

3. The subassembly of claim 2, wherein the portion of the body of the micro-electrode includes a sinusoidal shape that deviates from the axis, wherein the one or more portions of the portion of the body of the micro-electrode are defined by peaks of the sinusoidal shape.

4. The subassembly of claim 1, wherein the micro-electrode is formed from one or more conductive filaments including one or more of a combination of a metal material and a non-metal material.

5. The subassembly of claim 4, wherein the metal material includes one or more of stainless steel, carbon, tungsten, platinum and iridium.

6. The subassembly of claim 4, wherein the non-metal material includes a conductive polymer.

7. The subassembly of claim 1, wherein the outer diameter of the micro-electrode ranges between approximately 12.5 m-50 m, wherein the length of the micro-electrode ranges between approximately 10 mm-40 mm.

8. A spatially-adjustable animalia-engaging portion of a brain-computer interface, that spatially-adjustable animalia-engaging portion comprising:
a micro-electrode retainer portion including a distal end, a proximal end and a length extending between the distal end of the micro-electrode retainer portion and the proximal end of the micro-electrode retainer portion, wherein the micro-electrode retainer portion further defines
at least one spatial-adjuster-component-containing passage extending through the length of the micro-electrode retainer portion,
at least one spatial-adjuster-guide-post passage extending through the length of the micro-electrode retainer portion, and
at least one tube-and-micro-electrode-containing passage extending through the length of the micro-electrode retainer portion;
a micro-electrode guide portion including a distal end, a proximal end and a length extending between the distal end of the micro-electrode guide portion and the proximal end of the micro-electrode guide portion, wherein the micro-electrode guide portion further defines
at least one spatial-adjuster-component-containing passage extending through the length of the micro-electrode guide portion,
at least one spatial-adjuster-guide-post passage extending through the length of the micro-electrode guide portion, and
at least one micro-electrode-containing passage extending through the length of the micro-electrode guide portion;
at least one spatial-adjuster-component disposed within the at least one spatial-adjuster-component-containing passage of each of the micro-electrode retainer portion and the micro-electrode guide portion;
at least one spatial-adjuster-guide-post disposed within the at least one spatial-adjuster-guide-post passage of each of the micro-electrode retainer portion and the micro-electrode guide portion; and
at least one subassembly of claim 1 disposed within the at least one tube-and-micro-electrode-containing passage of the micro-electrode retainer portion, wherein an intermediate portion of the length of the micro-electrode of the at least one subassembly that extends beyond the distal end of the micro-electrode retainer portion is arranged within the at least one micro-electrode-containing passage of the micro-electrode guide portion, wherein a distal portion of the length of the micro-electrode of the at least one subassembly extends beyond the distal end of the micro-electrode guide portion.

9. The spatially-adjustable animalia-engaging portion of claim 8, wherein a proximal portion of the length of the micro-electrode of the at least one subassembly extends between the distal end of the micro-electrode retainer portion and the proximal end of the micro-electrode guide portion.

10. The spatially-adjustable animalia-engaging portion of claim 8, wherein the at least one spatial-adjuster-component-containing passage of the micro-electrode guide portion is defined by a threaded surface that is interfaced with an outer threaded surface of the at least one spatial-adjuster-component.

11. The spatially-adjustable animalia-engaging portion of claim 8, wherein the distal end of the micro-electrode retainer portion is arranged in a spaced-apart opposing relationship with respect to the proximal end of the micro-electrode guide portion.

12. The spatially-adjustable animalia-engaging portion of claim 8, wherein the at least one spatial-adjuster-component-containing passage of each of the micro-electrode retainer portion and the micro-electrode guide portion are axially-aligned, wherein the at least one spatial-adjuster-guide-post passage of each of the micro-electrode retainer portion and the micro-electrode guide portion are axially-aligned.

13. The spatially-adjustable animalia-engaging portion of claim 8, wherein the at least one tube-and-micro-electrode-containing passage of the micro-electrode retainer portion is axially-aligned with the at least one micro-electrode-containing passage of the micro-electrode guide portion.

14. A computing resource interface portion of a brain-computer interface, the computing resource interface portion comprising:
at least one interface subassembly including a distal biased pin, an intermediate biasing member and a proximal electrical contact; and
a micro-electrode retainer interface body portion defined by a length extending between a distal end of the micro-electrode retainer interface body portion and a proximal end of the micro-electrode retainer interface body portion, wherein the micro-electrode retainer interface body portion includes an inner surface that defines at least one biased-pin-containing passage extending through the length of the micro-electrode retainer interface body portion, wherein access to the at least one biased-pin-containing passage is provided by a distal opening formed by the distal end of the micro-electrode retainer interface body portion and a proximal opening formed by the proximal end of the micro-electrode retainer interface body portion, wherein the at least one interface subassembly is disposed within the at least one biased-pin-containing passage and arranged adjacent one or more portions of the inner surface of the micro-electrode retainer interface body portion.

15. The computing resource interface portion of claim 14, wherein the distal biased pin includes
a body extending between a distal end of the body of the distal biased pin and a proximal end of the body of the distal biased pin, wherein the intermediate biasing member includes
a body extending between a distal end of the body of the intermediate biasing member and a proximal end of the body of the intermediate biasing member, wherein the distal end of the body of the intermediate biasing member is disposed adjacent the proximal end of the body of the distal biased pin, wherein the proximal electrical contact includes
a body extending between a distal end of the body of the proximal electrical contact and a proximal end of the body of the proximal electrical contact, wherein the distal end of the body of the proximal electrical contact is disposed adjacent the proximal end of the body of the distal biased pin.

16. The computing resource interface portion of claim 15, wherein the proximal electrical contact is fixed adjacent the inner surface of the micro-electrode retainer interface body portion.

17. The computing resource interface portion of claim 15, wherein a portion of a length of the proximal electrical contact extends through the proximal opening and beyond the proximal end of the micro-electrode retainer interface body portion.

18. The computing resource interface portion of claim 15, wherein the distal biased pin is movably-disposed within the at least one biased-pin-containing passage.

19. The computing resource interface portion of claim 15, wherein the intermediate biasing member biases a shoulder surface of the distal biased pin adjacent a portion of the one or more portions of the inner surface of the micro-electrode retainer interface body portion defining a ledge surface such that a portion of a length of the distal biased pin extends through the distal opening and beyond the distal end of the micro-electrode retainer interface body portion.

20. The computing resource interface portion of claim 15, wherein the body of the distal biased pin defines
an axial passage extending between the distal end of the body of the distal biased pin and the proximal end of the body of the distal biased pin, wherein the body of the proximal electrical contact defines
an axial passage extending between the distal end of the body of the proximal electrical contact and the proximal end of the body of the proximal electrical contact, wherein the body of the intermediate biasing member defines
an axial passage extending between the distal end of the body of the intermediate biasing member and the proximal end of the body of the intermediate biasing member, wherein the axial passages collectively define
at least one micro-electrode access passage.

21. A brain-computer interface comprising:
the spatially-adjustable animalia-engaging portion of claim 8; and
the computing resource interface portion of claim 14 connected to the spatially-adjustable animalia-engaging portion, wherein the computing resource interface portion further includes an actuator that is connected to the at least one spatial-adjuster-component of the spatially-adjustable animalia-engaging portion that is configured to rotate the at least one spatial-adjuster-component for further extending the distal portion of the length of the micro-electrode of the at least one subassembly of the spatially-adjustable animalia-engaging portion beyond the distal end of the micro-electrode guide portion of the spatially-adjustable animalia-engaging portion.

22. The brain-computer interface of claim 21, wherein upon connecting the computing resource interface portion to the spatially-adjustable animalia-engaging portion, the distal end of the body of the distal biased pin of the interface subassembly of the computing resource interface portion is disposed adjacent at least one of the proximal surface of the micro-electrode-containing tube and the proximal surface of the at least one micro-electrode of the at least one subassembly for electrically-connecting a distal end of the computing resource interface portion to a proximal end of the spatially-adjustable animalia-engaging portion.

23. The brain-computer interface of claim 22, wherein the proximal end of the body of the proximal electrical contact of the interface subassembly of the computing resource interface portion is connected to a conduit for connecting the computing resource interface portion to a computing resource.

24. The brain-computer interface of claim 23, wherein the conduit is a wired conduit that hard-wire connects the computing resource interface portion to a computing resource.

25. The brain-computer interface of claim 23, wherein the conduit is a wireless conduit that wirelessly connects the computing resource interface portion to a computing resource.

26. The brain-computer interface of claim 21, wherein the computing resource interface portion further includes
at least one male connector portion extending away from the distal end of the body portion of the computing resource interface portion, wherein the spatially-adjustable animalia-engaging portion defines
at least one female connector portion extending into the proximal end of the micro-electrode retainer portion that is sized for receiving the at least one male connector portion for connecting the computing resource interface portion to the spatially-adjustable animalia-engaging portion.
